(19) United States
(12) Patent Application Publication
(10) Pub. No.: US 2006/0094004 A1
(43) Pub. Date: May 4, 2006

(54) Micro-reactor, biological material inspection device, and microanalysis system

(76) Inventors: Akihisa Nakajima, Sagamihara-shi (JP); Eiichi Ueda, Tokyo (JP); Kusunoki Higashino, Osaka (JP); Yasuhiro Sando, Amagasaki-shi (JP); Nobuhisa Ishida, Kyoto-shi (JP); Youichi Aoki, Amagasaki-shi (JP)

Correspondence Address: CANTOR COLBURN, LLP, 55 GRIFFIN ROAD SOUTH, BLOOMFIELD, CT, 06002, US

(21) Appl. No.: 11/255,664

(22) Filed: Oct. 21, 2005

(30) Foreign Application Priority Data
Oct. 28, 2004 (JP) JP2004-314244
Feb. 7, 2005 (JP) JP2005-030615
Feb. 8, 2005 (JP) JP2005-031748
Mar. 31, 2005 (JP) JP2005-103525

(51) Int. Cl. C12Q 1/70

(52) U.S. Cl. 435/5

(57) ABSTRACT

A micro reactor for inspecting a biological material, comprising: a first substrate on which a minute flow path is formed; a second substrate laminated on the first substrate so as to cover the minute flow path; a detection section provided on the minute flow path formed between the first and second substrates so as to inspect a liquid mixture of a sample and a reagent; (4) an opening section formed at a region on the second substrate corresponding to the detection section so as to irradiate inspection light to the liquid mixture arriving the detection; and (5) a transparent member to cover the opening section.

This application is based on Japanese Patent Application No. 2004-314244 filed on Oct. 28, 2004, Japanese Patent Application No. 2005-030615 filed on Feb. 7, 2005, Japanese Patent Application No. 2005-031748 filed on Feb. 8, 2005, Japanese Patent Application No. 2005-103525 filed on Mar. 31, 2005 in Japanese Patent Office, the entire content of which is hereby incorporated by reference.

### BACKGROUND OF THE INVENTION

The invention relates to a micro reactor for biological material, a biological material inspection device including the micro reactor and a microanalysis system.

In recent years, there have been developed systems in which making full use of micro machine technologies and ultra fine machining technologies, a system integrated and miniaturized existing devices and means for sample preparation, chemical analysis and chemical synthesizing (e.g. pump, valve, flow path and sensor) on a chip. This is called μ-TAS (Micro total Analysis System), bioreactor, lab-on-chips or biochips and its application is expected in the fields of medical inspection and diagnosis, environment measurement, and agricultural production. In particular, when complicated processes, skilled handwork and operations of machines and instruments are required, as seen in genetic inspection, a micro reactor which is a micro analysis system makes analysis automatic, speeding up and simple, enormously provides benefits in terms of not only cost, amount of necessary sample and consumed time but also capability of analysis regardless of time and place.

In the medical site where various inspections including clinical tests are carried out, quantitative character and accuracy of analysis are regarded to be important even in the measurement by a chip type micro reactor, which rapidly outputs results regardless of place. It is a subject to establish a highly reliable fluid feeding system with simple structure because analyzer chip has severe restrictions in terms of size and shape. Therefore, a micro fluid control element having high accuracy-and reliability is desired. The inventers of the invention already have suggested a micro pump system preferable for this purpose (Patent Documents 1 and 2).

Also, there has been suggested a microanalysis system capable of automatically analyze information by set an inspection chip in a inspecting device by employing such a micro pump system.

In a comprehensive microanalysis system, for example, an inspection chip is configured in such a way that a sample to be tested and a regent for detecting the information on this sample are separately stored in-different sites, wherein the sample storage section communicates with the reagent storage section through a liquid path, and the sample and reagent are mixed in this communicating path to perform effective reaction, while being fed downstream.

As described above, in a comprehensive microanalysis system, composed of an inspection chip and a fluid control/detection apparatus, capable of automatic detection of required information, an operating fluid is fed to the inspection chip from the fluid control/detection apparatus through a micro-pump as appropriate. As the operating fluid is fed, the sample and reagent are mixed so that reaction takes place at a reaction site whose temperature is set to a predetermined level. At the detection section, required information is obtained by an adequate device.

The aforementioned detection device is defined as a device wherein measurement light is applied to the site to be detected on the analysis flow path, from a LED or the like for each inspection item, so that transmitted light or reflected light is detected by an optical detection device such as a photodiode and photomultiplier tube. The optical detection device includes various types of apparatuses based on different principles. Of these apparatuses, the ultraviolet-visible spectrophotometer is preferably used.

The comprehensive microanalysis system using the optical detection device to perform detection is required to ensure higher efficiency and higher precision in detecting the inspection chip containing a biological substance and a reagent in advance.

In the conventional inspection chip, the light emitted from the ultraviolet-visible spectrophotometer and others is applied to the object to be tested, through the optically transparent resin constituting the laminate of the inspection chip. Accordingly, even if the substrate is made of plastic, noise tends to be produced from the light reflected from the interface with the plastic or the light having passed through it obliquely. Thus, the prior art has been required to provide more accurate information.

The present invention has been made to solve the aforementioned problems involved in the prior art. The object of the present invention is to provide a comprehensive microanalysis system for automatic detection of required information on a test object, this information being contained in an inspection chip, wherein an optical detection device is employed to ensure more accurate detection.

In a micro reactor in the above microanalysis system, since fine flow paths and flow path elements are provided on a small plate, it is necessary that the character of a fluid, for instance, the viscosity is controlled within an appropriate range, so that the fluid may flow smoothly, then is divided, mixed and reacted efficiently in a micro space. It is preferred that this is adjusted in a preliminary treatment step of the specimen.

Also, for a chip handing many of specimen, in particular, a chip handling clinical specimen having risk of pollution and infection, disposability is desired and issues such as versatility and mass productivity have to be overcome.

For the materials constituting micro reactor chip, selection of the materials is wide i.e. glass, ceramic, silicon, various kinds of plastics and metal, and a variety of materials can be used as occasion. Workability, chemical resistance, heat resistance and inexpensiveness are required when the material is selected. As there is no superior material fulfilling all the requirements, appropriated selection of material is desired, considering chip structure, usage and method of detecting. For example, silicon, in which the working technologies such as photolithography and etching grown in semiconductor production technologies can be applied, has demerits of opacity and high price. The glass, a material having transmittance state and heat resistance, has problems that biological material is absorbed nonspecifically and its workability is not always preferable. Thus, chips in which a plurality of materials appropriately combined are also manufactured.

As mentioned above, for a micro reactor that provides simple and rapid inspection method, specific problems and desires to be solved and requests are still raised, and solutions of them are desired.

(Patent Document 1) Unexamined Japanese Patent Application No. 2001-322099

(Patent Document 2) Unexamined Japanese Patent Application NO. 2004-108285

(Non-patent Document 1) (DNA Chip Technologies and Its Applications), (Protein, nucleic acid, enzyme) Vol. 43, issue 13 (1998) by Fusao Kimizuka, Ikunoshin Kato, issued by Kyoritsu Publication Co., Ltd.

### DISCLOSURE OF THE INVENTION

### Problems to be Solved by the Invention

Then, the invention suggests a micro reactor for biological material inspection that can analyze highly sensitively by a chip that is made of resin, which is a superior material in workability and inexpensiveness, and has a waste fluid reservoir and preliminary treatment means. Further, the micro reactor is made to be of a disposable type to make it to be an analyzing tool with less risk of pollution and infection. Furthermore, an object of the invention is to provide a biological material inspection device wherein a simple structure and a highly accurate fluid feeding system are incorporated and highly accurate analysis can be carried out for at least one inspection item.

### SUMMARY OF THE INVENTION

The above objects can be attained by the following structures.

A micro reactor for inspecting a biological material, comprising:

(1) a first substrate on which a minute flow path is formed;

(2) a second substrate laminated on the first substrate so as to cover the minute flow path;

(3) a detection section provided on the minute flow path formed between the first and second substrates so as to inspect a liquid mixture of a sample and a reagent;

(4) an opening section formed at a region on the second substrate corresponding to the detection section so as to irradiate inspection light to the liquid mixture arriving the detection section; and

(5) a transparent member to cover the opening section.

In the above structure, the detection section comprises a land section provided to reduce the depth of the minute flow path.

Further, a biotinophilic protein to trap gene amplified in the minute flow path is adsorbed on the detection section.

### BRIEF DESCRIPTION OF THE DRAWINGS

FIG. 1 is a perspective view representing the comprehensive microanalysis system as an embodiment of the present invention;

FIG. 2 is a schematic view representing an inspection chip and an inspection method used in the comprehensive microanalysis system shown in FIG. 1; and

FIG. 3 is a schematic cross sectional view representing the structure of the inspection chip of the inspection section shown in FIG. 2.

FIG. 4 is a schematic drawing indicating a biological material inspection device constituted of a micro reactor and a device main body.

FIG. 5 is a schematic drawing indicating a micro reactor for biological material inspection of the First Embodiment of the invention.

FIG. 6 is a cross-sectional view of a micro reactor. It indicates a flow path starting from a reagent containing section, specimen preliminary treatment section 20a in FIG. 7 and a positional relation of a merging from a sample port 19. Meanwhile, the elements shown by dotted lines are not in the same cross-section as the elements shown by solid lines. Also, specimen treatment fluid containing section 20b is not indicated in this drawing.

FIG. 7 shows a cross-sectional view at a position where minute flow paths intersect perpendicularly in a micro reactor that is constituted of four pieces of substrates.

FIG. 8(a) is a cross-sectional view indicating an example of a piezo pump. FIG. 8(b) is a top view of it. FIG. 8(c) is a cross-sectional view indicating other example of a piezo pump.

FIG. 9(a) and FIG. 9(b) are cross-sectional views indicating an example of a check valve located in a flow path.

FIG. 10 shows cross-sectional views of an example of an active valve located in a flow path wherein FIG. 10(a) indicates an open status and FIG. 10(b) indicates a close status respectively.

FIG. 11 is a drawing indicating a structure of reagent dividing and a reagent mixing section in a micro reactor of an embodiment of the invention.

FIG. 12 is a drawing indicating specimen containing section 20, specimen preliminary treatment section 20a and specimen dividing.

FIG. 13 is a perspective view showing a polystyrene sheet having a silicone rubber with holes.

### DETAILED DESCRIPTION OF THE PREFERRED EMBODIMENT

Firstly, preferable structures to attain the above objection are explained hereinafter. Meanwhile, in this description, “substrate” means a member in which fine working and modification on the scale of 0.1 μm to several millimeters are applied as in micro array or DNA chip. “Flow path element” is a functional parts installed in micro reactor. “Minute flow path” is a fine-channel-shaped flow path formed on micro reactor substrate of the invention. “Gene” means DNA or RNA which carries genotypic information that creates some function, however, it may also mean chemical substance in the type of DNA and RNA. The substances to be analyzed may sometimes be called “analyte”. And, “Base” denotes a nucleic acid base of nucleotide.

The comprehensive microanalysis system of the present invention for achieving the aforementioned object comprises:

a fluid path formed by micromachining; and

an inspection chip proper composed of an optically transparent first substance and a second substrate laminated so as to cover the micromachined surfaces of the first substrate;

wherein a liquid mixture of sample and reagent inside are fed to the minute flow path for supplying a fluid formed between the first and second substrates, a detection section is arranged in the middle of the minute flow path; the comprehensive microanalysis system applies light to the liquid mixture having reached the detection section; and automatically inspects the information on the object to be tested by using this light;

wherein an opening is formed in the area corresponding to the detection section of the second substrate, and this opening is covered with a transparent member or a quasi-transparent film.

The present invention of the aforementioned structure improves detection accuracy because the portion serving as an optical path does not contain any part that may interfere application of light.

Further, in the present invention, at least the outer periphery of the opening of the second substrate is colored, and heat is applied to this colored area from the film side by a laser, whereby the film is heat-sealed with the first substrate.

This structure allows the film to be easily bonded.

Further, in the present invention, the minute flow path constituting the detection section is provided with a land to reduce the depth of the minute flow path.

The present invention characterized by the aforementioned structure provides the following advantage. For example, a biological substance is inspected using the inspection chip. In this case, if a biotinophilic protein such as streptoavidin is adsorbed on the upper portion of the land, then the biotinophilic protein specifically bonds with the biotin labeled with the 5′-terminus of the primer used in the reaction of gene amplification reaction. This arrangement allows the biotin-labeled probe or amplified gene to be trapped by the detection site with a high degree of sensitivity.

The comprehensive microanalysis system of the present invention ensures high-precision detection to be performed at the detection section.

Further, the portion surrounding the opening is colored. This allows the film to be heat-sealed by a laser.

In addition, a land is arranged inside the flow path constituting the detection section. This arrangement minimizes the noise involved in detection and improves the sensitivity in detection.

The invention is a chip type micro reactor in which a main body is constructed by at least two substrates, which is represented by a micro reactor for biological material inspection in which a micro pump port, a valve base and a waste fluid reservoir are formed as structural members, by using a channel-formed substrate and a covering substrate, and minute flow paths are formed on the cannel-formed substrate, and at least these structural members, the minute flow path and the detecting section on the channel-formed substrate are covered by a light transmitting covering substrate that is brought into close contact with them.

Aforesaid substrates are preferably polystyrene, a PE, polypropylene, a polyethylene terephthalate, polyethylenenaphthalate, polymethylmethacrylate, polyethylene vinyl alcohol, acryl resin, polyvinyl resin, epoxy-resin, polyvinyl chloride, a unsaturated polyester resin, polyamide resin, polyimide resin, polysulfone resin, annular cycloolefin resin, cellulose acetate, cellulose nitrate, fluorocarbon resin, polycarbonate, or poly dimethyl siloxane.

Also aforesaid channel-formed substrate is preferably a resin in polystyrene type. The inner surface of aforesaid minute flow path is preferably coated by protein. Aforesaid minute flow path is the minute flow path branched into at least two or more paths, Through a micro pump and a feeding fluid dividing means, a fluid containing specimen is fed to downstream point in each branched minute flow path and/or a reagent encapsulated or its mixture fluid are fed to downstream points in each branched minute flow path. At downstream points of each branched minute flow path, a plurality of items and/or controls can be measured simultaneously.

Aforesaid micro pump is a piezo pump provided with a first flow path whose flow resistance varies with the change of pressure difference, a second flow path in which a ratio of a change of flow resistance to a change of pressure difference is smaller than that of the first flow path, a pressure chamber connected to both the first flow path and the second flow path, and with an actuator which changes the pressure inside the pressure chamber.

Also, feeding fluid dividing means including branched minute flow paths, a feeding fluid control section able to control the passage of the fluid though the pump pressure of aforesaid micro pump that intercepts passage of the fluid until the fluid feeding pressure of positive direction reaches to a predetermined pressure and allows passage by applying fluid feeding pressure exceeding the predetermined pressure, and a backward flow preventing section which prevents backward flow in the flow path, controls feeding of feeding fluid, an fixed amount of feeding fluid and mixing of each fluid in the divided flow path.

It is also a micro reactor in which a substrate is further adhered as a bottom substrate, to cover a concave formed on the bottom of aforesaid channel-formed substrate so as to form a waste fluid reservoir section to reserve the waste fluid resulting from condensing, washing and measuring of the specimen.

It is preferable that the viscosity of fluid flowing through aforesaid minute flow path is controlled to be not more than 10 mPa·s. The invention is a biological material inspection device composed of a device main body in which a detecting device which optically detects biological material contained in specimen, a micro pump, a control device for the micro pump and a temperature control device are united together, and of a micro reactor for aforesaid biological material inspection wherein biological material is measured automatically when the micro reactor is mounted on device main body.

A biological material inspection device of the invention has a system structure wherein a chip component constructed from micro reactors for each specimen equipping elements for reagents and for fluid feeding system, and a control and detection component that is a device main body are separated. Thereby, cross contamination and carryover contamination hardly occur for micro analysis and amplification reaction.

The chip of micro reactor of the invention is made of resin, which is a superior material in workability and is of suitable for mass production including the material and constituent element, and can be produced at low cost since probes and regents to be used for detection are easily obtained. The biological material inspection device and micro reactor of the invention are capable of measuring multiple items simultaneously and has a versatility to cope with diversification of purposes.

The following describes another embodiment of the present invention:

The present invention provides a microreactor for gene inspection containing a micro-pump element and a minute flow path, wherein at least the detection portion is a microreactor made of transparent plastic, and the biotinophilic protein for trapping the gene amplified inside the minute flow path is absorbed, as a detection site, downstream of the amplification reaction site of the minute flow path.

The aforementioned biotinophilic protein binds with the biotin labeled to the 5′-terminus of the primer used for amplification reaction of a sample gene.

The aforementioned biotinophilic protein is preferably streptoavidin.

The aforementioned streptoavidin is preferably adsorbed by applying the solution obtained by dissolving in a buffer solution, to the portion for immobilization inside the minute flow path.

The aforementioned streptoavidin solution preferably has a concentration of 10 through 35 μg/mL.

The above-mentioned buffer solution preferably is a physiological saline solution or an SSC buffer.

The above-mentioned amplification reaction is preferably performed according to the ICAN (registered trademark) method.

The probe to be hybridized with the amplified gene is labeled with fluorescent pigment.

The above-mentioned fluorescent pigment preferably is a FITC.

The probe is preferably colored with gold colloid modified by the anti-FITC antibody.

In order to incorporate a DNA amplification manufacturing process in advance of detection, gene analysis with high sensitiveness is possible with the micro reactor of the present invention, and since the probe DNA which carries out hybridization specifically for the amplified gene is used, it can detect the target gene with preferable accuracy.

Moreover, since the micro reactor of the present invention is a disposable type chip, serious problems, such as crossing contamination and carryover contamination, do not occur easily for a microchemical analysis and an amplification reaction. Since washing removal of nonspecific joint materials other than the combination (or interaction) of sample DNA and a primer and a probe is easy, a micro reactor of a lower back ground can be offered.

The micro reactor of the present invention has a structure that it including material and a structural element is fit for quantity production, and, moreover, it can be used for multi item analysis. Since the trap and detection methods for the amplified target gene are an already established simple technique, and moreover since the probe and regents used for detection, and the thing which chemicals can be obtained easily, the micro reactor of the present invention can be manufactured at low cost.

The present invention provides a microreactor for gene inspection containing a minute flow path, wherein at least the detection portion is a microreactor made of transparent plastic, and the biotinophilic protein is absorbed by polystyrene, as a detection site, downstream of the amplification reaction site of the minute flow path, for the purpose of trapping the sample gene amplified by a biotin labeled primer inside the minute flow path.

The probe hybridized with the amplified gene is preferably labeled with peroxidase.

Further, the probe is made to react with a coloring reagent.

The above-mentioned coloring reagent as a coloring substance contains 3,3′,5,5′-tetramethyl benzidine, 3,3′-diamino benzidine, p-phenylene diamine, 5-amino salicylic acid, 3-amino-9-ethylcarbazole, 4-chloro-1-naphthol, 4-amino antipyrine or o-dianisidine.

The present invention provides a DNA inspection device containing:

a microreactor for gene inspection;

a micro-pump;

an apparatus for controlling the micro-pump and temperature; and

a detection apparatus for optical detection of gene amplification reaction;

wherein, when the microreactor is mounted, gene amplification reaction and detection of gene amplification reaction is automatically carried out.

The above-mentioned microreactor is a piezo-pump provided with:

a first flow path whose resistance changes in response to differential pressure;

a second flow path wherein the ratio of the change in flow path resistance in response to differential pressure is smaller than that of the first flow path;

a pressure chamber connected to the first and second flow paths; and

an actuator for changing the pressure inside the pressure chamber.

The above-mentioned detection apparatus is characterized in that:

after the gene amplified inside the minute flow path has been bonded with the biotinophilic protein adsorbed on the detection site of the minute flow path, a hybridized product is obtained by hybridization with the probe DNA; or

after the hybridized product obtained by hybridization between the gene amplified inside the minute flow path and the probe DNA has been bonded with the biotinophilic protein adsorbed on the detection site of the minute flow path, the probe DNA is converted into a coloring substance;

wherein the above-mentioned detection apparatus performs optical detection subsequent to following one of the above-mentioned steps.

The method of creating a detection site of the microreactor for gene inspection according to the present invention is characterized in that, in order to ensure that the biotinophilic protein for trapping the gene amplified inside the minute flow path is immobilized, as a gene detection site, downstream of the gene amplification reaction site of the flow path, the biotinophilic protein is dissolved in a physiological saline solution or an SSC buffer to prepare a solution having a concentration of 10 through 35 μg/mL, and the solution is applied to the minute flow path formed with polystyrene, whereby biotinophilic protein is adsorbed on the minute flow path.

The method of detecting the amplified gene of the present invention includes the steps of:

modifying the amplified gene into one chain by mixing between the reaction solution containing the gene amplified at the gene amplification reaction site in the minute flow path of the microreactor, and the modification solution;

feeding the processing solution obtained by modification of the amplified gene into one chain, to the inspection site of the minute flow path wherein the biotinophilic protein is adsorbed onto the polystyrene so that the amplified gene is trapped by the detection site;

feeding the peroxidase-modified probe DNA to the detection site so as to hybridize the amplified gene with the probe DNA;

feeding the coloring reagent solution containing the coloring substance to the detection site trapping the amplified gene so that the coloring substance will be colored by the reaction catalyzed by peroxidase; and

optically measuring the coloring that takes place at the detection site.

The microreactor of the present invention is capable of highly sensitive gene analysis for incorporating the DNA amplification process prior to detection, and provides high-precision detection of the target gene because the microreactor uses the probe DNA that is hybridized specifically with the amplified gene.

In the DNA inspection device of the present invention, a chip component for each sample, equipped with the elements for the reagent/liquid feed system, and a control/detection component as a DNA inspection device proper are arranged separately from each other. This structure ensures that microanalysis and amplification reaction is impervious to a serious impact of cross contamination carry-over and contamination. Further, it is easy to wash and remove the nonspecific bond other than the bondage (or interaction) of the sample DNA with the primer and probe. Thus, this structure provides a microreactor chip with a lower background.

The micro reactor of the present invention has a structure that it including material and a structural element is fit for quantity production, and, moreover, it can be used for multi item analysis. Since the trap and detection methods for the amplified target gene are an already established simple technique, and moreover since the probe and regents used for detection, and the thing which chemicals can be obtained easily, the micro reactor of the present invention can be manufactured at low cost.

The following describes the embodiment of the present invention with reference to drawing:

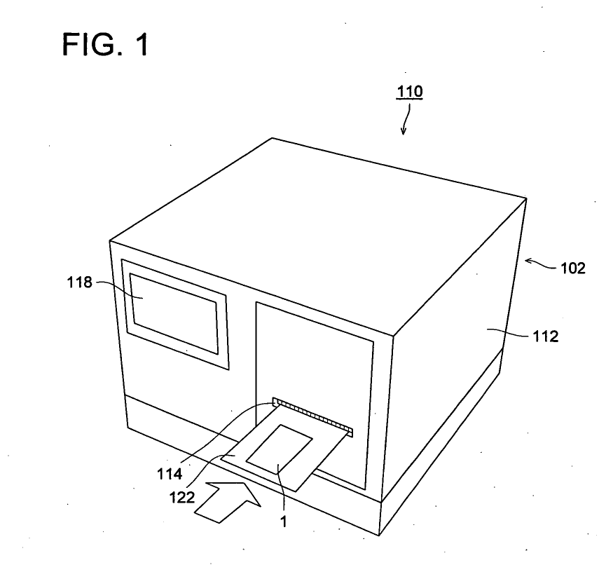

FIG. 1 is a comprehensive microanalysis system as an embodiment of the present invention.

The comprehensive microanalysis system 110 is composed of an inspection chip 1 (a micro-reactor) made of one resin-made chip; and a fluid control/inspection apparatus 102 for inspecting the required information by setting this inspection chip 1 at a predetermined position.

The inspection chip 1 is designed in such a way that, when the gene sample extracted from the blood or phlegm is injected therein, gene amplification reaction and detection are carried out automatically inside the chip according to ICAN method or others, and simultaneous diagnosis of a plurality of genes can be diagnosed. This inspection chip 1 has a length and width of several centimeters. The DNA of about 2 through 3 μL, for example, is dropped in this inspection chip 1, and the chip is then mounted inside the fluid control/inspection apparatus 102. This simple procedure alone ensures automatic amplification reaction and inspection to be carried out automatically.

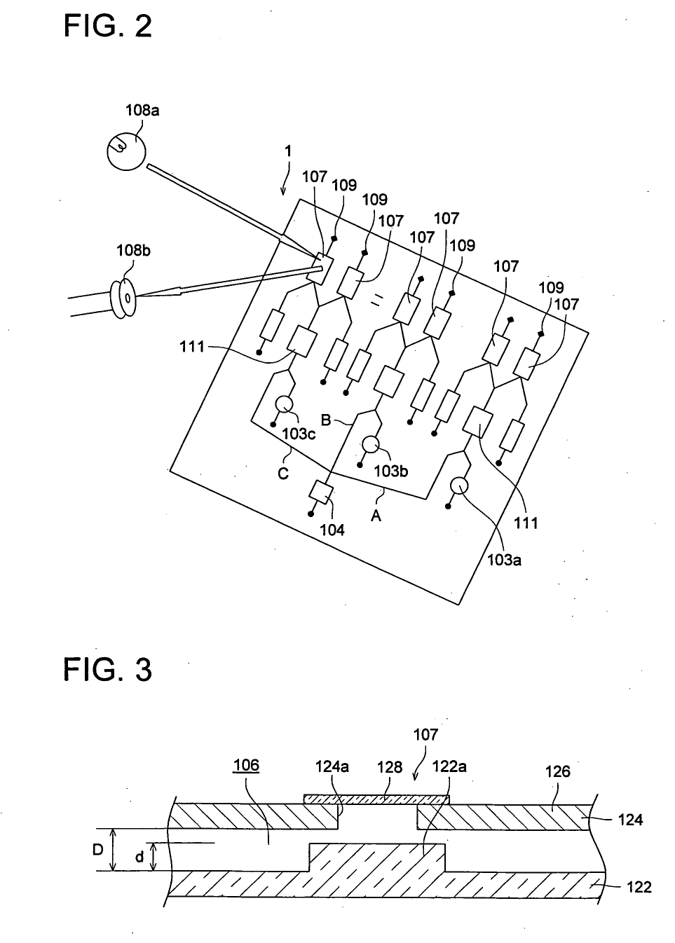

The structure of the aforementioned inspection chip 1 is schematically shown in FIG. 2.

To be more specific, the inspection chip 1 allows reagent to be fed out of the portion upstream of the DNA amplification reaction exposure section 104, by the operating fluid. After the reagent has diverged in three flow paths, the operating fluid is introduced from an opening arranged upstream of each of a sample storage section 103a with the sample stored therein, a positive control storage section 103b with positive control stored therein, and a negative control storage section 103c with negative control stored therein. The sample storage section 103a, positive control storage section 3b and negative control storage section 103c are pushed out by the operating fluid having been introduced, whereby the DNA in the sample, positive control and negative control are mixed with the aforementioned DNA amplification reagent. In an amplification reaction section 111, amplification reaction is performed at a predetermined temperature for a predetermined length of time. After that, each of them is divided into two parts, which are then fed to the detection section 107. The DNA having been amplified by the detection section 107 is immobilized here and is stained by the sample DNA amplification/detection reagent and internal control amplification/detection reagent. The degree of staining is checked, and a required information is obtained by an optical detection apparatus composed of a light-emitting device 8a and light receiving device 108b such as LED, for example, in such a way as to determine if the target DNA is present or not. An ejection section 109 communicating with the waste liquid storage section for storing the waste mixture subsequent to detection is formed downstream of the detection section 107. Each liquid path is provided with an air purge, a waste liquid valve for adjusting the timing of liquid merging, and a mechanism for discarding the leading portion of the liquid where mixing is unstable. This arrangement is provided to ensure a high-precision flow of liquid.

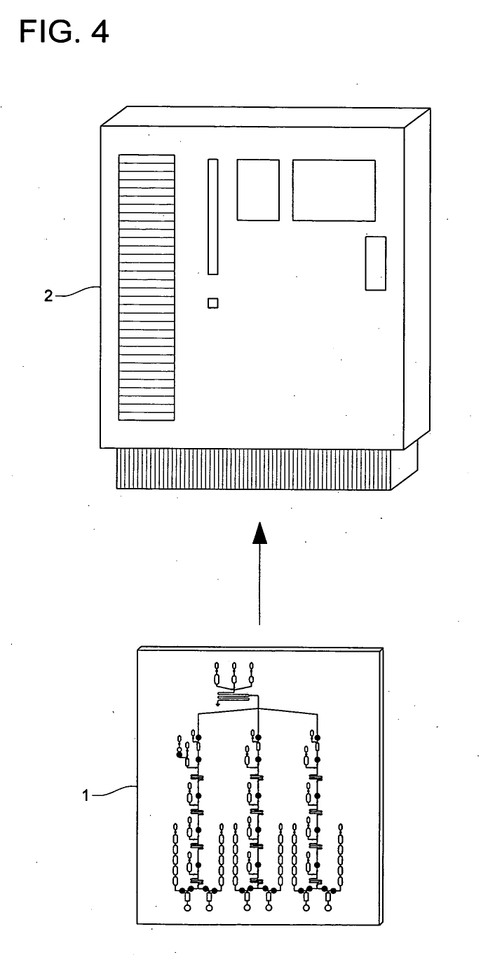

FIG. 3, schematically shows the structure of the fluid detection section 107 in particular, inside the minute flow path 106.

The minute flow path 106 arranged on the inspection chip 1 of the present invention is provided between the first substrate 122 having been micromachined and the second substrate 124 laminated so as to cover the micromachined surfaces of the first substrate 22. The middle portion of the minute flow path 6 is formed into the detection section 107.

Of the first substrate 122 and second substrate 124, at least the first substrate 122 provided with micromachining is made of a transparent member. The portion of the first substrate 122 corresponding to the detection section 107 has a protruding land 122a that reduces the depth of the detection section 107. This land 122a may be arranged in a rectangular, circular or trapezoidal shape. What is important is that the depth d of this portion is smaller than the normal depth D of the minute flow path 106. It goes without saying that the surface is preferably finished in a smooth shape.

As a material of the first base board 22, various shape-forming materials are available as a material of a chip, and it is used in accordance with each material characteristics. For example, fluorocarbon resin, such as polystyrene, polyethylene, polypropylene, a polyvinyl chloride, polycarbonate, and polytetrafluoroethylene, polysiloxane based polymers, such as poly dimethyl siloxane, polyolefin based polymers, such as polymethylmethacrylate, polyvinyl alcohol, and an ethylene-vinylalcohol copolymer, polyester based polymers, such as a polyethylene terephthalate and a polybutylene terephthalate, polyamide based polymers, such as 6-nylon, 6, and 6-nylon, a cellulose based polymer like an annular cycloolefin resin, polyarylate resin, a cellulose acetate or a cellulose nitrate, a various inorganic matter glass etc. may be listedded.

When fluorometry is used for detection, the fluorescent light of the plastic substrate per se constitutes the noise that raises a problem. A plastic substrate capable of removing such a noise has been proposed (Official Gazette of Japanese Patent Tokkai 2003-130874). It is also possible to use the plastic of low fluorescence intensity such as straight chain polyolefin, cyclic polyolefin and fluorine-containing resin.

It should be noted, however, that the present invention is not restricted thereto.

The second substrate 124 can be made of the same material as that of the first substrate 122. An opening 124a is formed in the area corresponding to the detection section 107. This opening 124a is not restricted to the circular or rectangular shape. Further, in contrast to the first substrate 122 made of a transparent member, the second substrate can be made in any color just as long as the outer periphery of the opening 124a is formed in colors; there is no restriction to the color of the second substrate. The size of the aforementioned colored portion is not restricted as long as it is greater than that of the opening 124a. To be more specific, the opening 124a in the present invention is covered with a transparent member or a quasi-transparent film 128. Accordingly, when a light beam is applied to this film 128, the film is required only to receive this light beam and to convert it into heat. Thus, the size of the colored portion should be determined properly in conformity to the size of the film.

The film 128 is preferably transparent or quasi-transparent. If the film is not colored, any resin can be used.

The detection section 107 of the present invention has the structure described above.

The detection apparatus (not illustrated) installed on the detection section 107 is used to detect the transmitted light through an optical detection device such as a photodiode and photomultiplier, when measurement light is applied, for example, from a LED to the detection site on the analysis flow path for each inspection item. The optical detection device includes various types of apparatuses based on different principles. Of these apparatuses, the ultraviolet-visible spectrophotometer is preferably used. It can be incorporated in the aforementioned inspection device or can be a separate apparatus, which is connected when used.

When detecting the required information on the object to be tested, this information having been supplied to the detection section 107 by the detection device, the measurement light such as that from the LED passes through the film 128 composed of a transparent member. The transmitted light passing through the land 122a is detected. This arrangement minimizes the entry of noise and improves the result of detection. Further, if the land 122a is coated with a biotin-binding protein such as streptoavidin (avidin, streptoavidin, extraavidin (R), and preferably streptoavidin) in advance, then the land 122a is specifically bonded with the biotin labeled with the probe substance or the biotin labeled with 5-terminus of the primer used for gene amplification reaction. This arrangement allows the biotin-labeled probe or amplified gene to be effectively trapped by this detection site. As described above, the land 122a improves the sensitivity.

A micro reactor of the invention for biological material inspection and a biological material inspection device including a micro reactor, a micro pump, various control devices and a detecting device are described specifically as follow:

In the micro reactor of the invention, respective elements or structural sections such as each storage section, flow path, pump connecting section, fluid feed control section, backward flow preventing section, reagent amount determination section, mixing section and waste fluid reservoir section are allocated in adequate positions functionally by micro working technologies.

Material of Chip

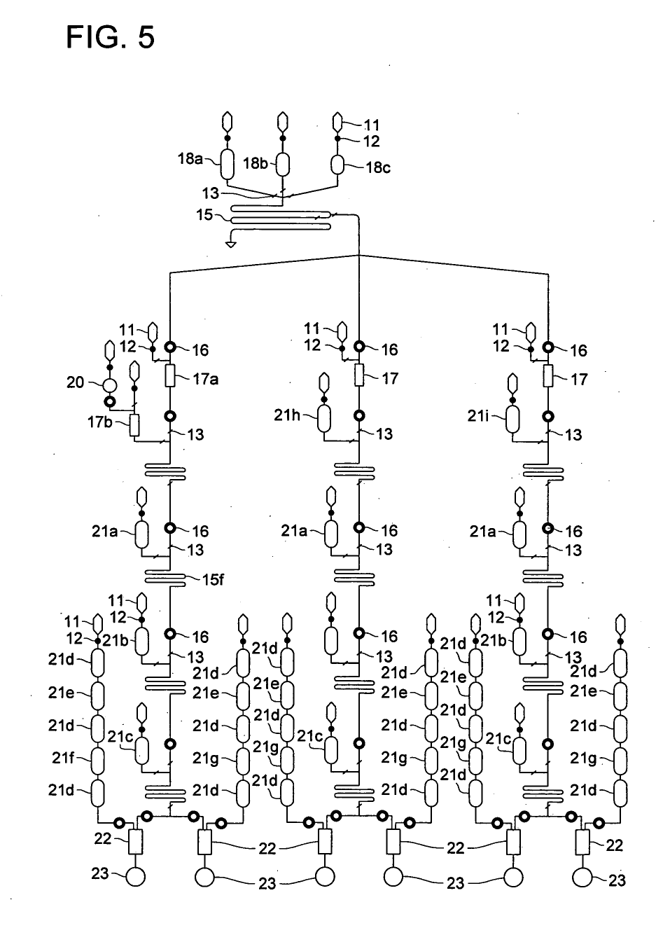

Micro reactor 1 shown in FIG. 4 and FIG. 5 is a piece of chip which is manufactured by adequately combining one or more forming material including e.g., resin, glass, silicon and ceramic. The invention is a chip type micro reactor in which a main body is constructed by at least two substrates. It has a specific structure in which, a micro pump port, a valve base and a waste fluid reservoir are formed as structural members by using a channel-formed substrate and a covering substrate as a basic structure, minute flow paths are formed at least on the cannel-formed substrate, and at least these structural members, the minute flow path and the detecting section on the cannel formed substrate or at least the detecting section are covered by a light transmitting covering substrate that is brought into close contact with the minute flow path.

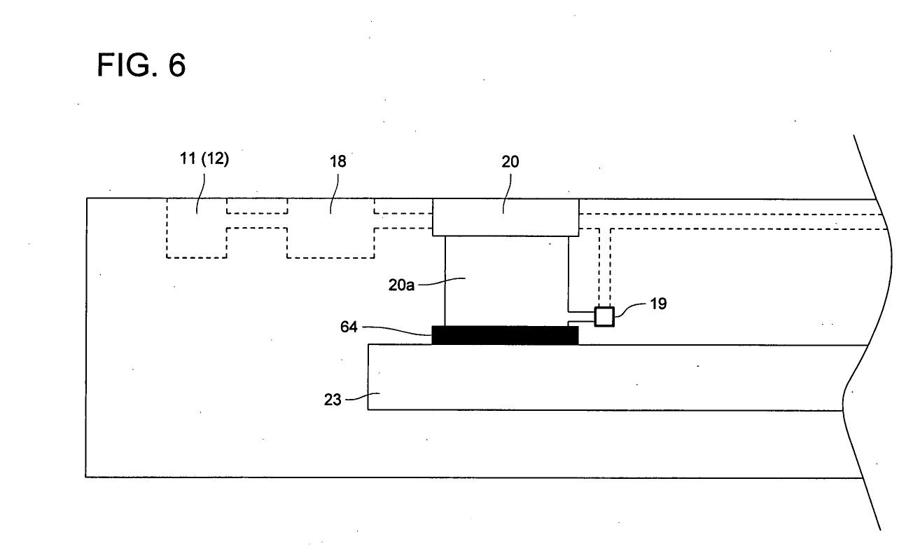

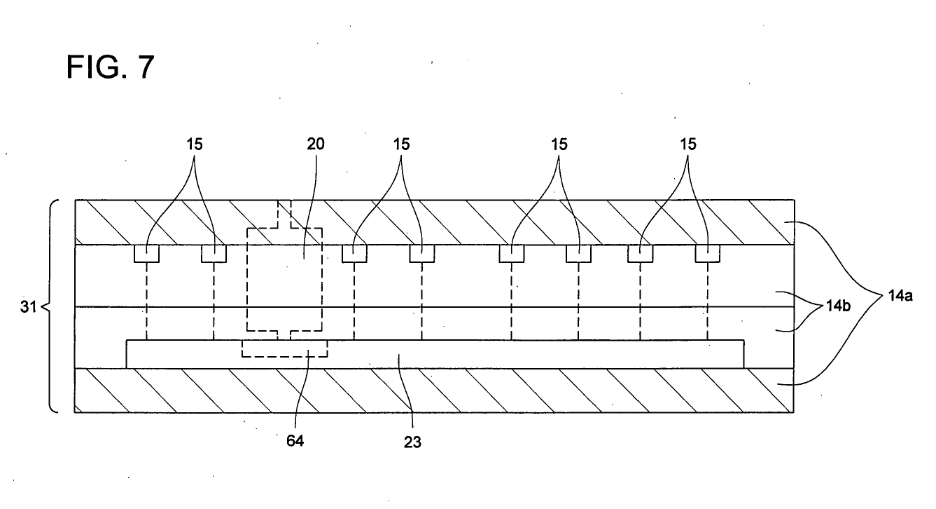

As explained afterward, when dividing specimen feeding fluid to feed the specimen into two or more flow paths of analysis and to merge with the reagent at the down stream, it is necessary to the form minute flow paths each having different height within the chip (FIG. 6). To realize the flow paths system, the flow path system needs to be formed by three layers of substrates by adding further a channel-formed substrate as an intermediate substrate. A multi layer build-up type chip shown in FIG. 7 is an example of it and at the bottom of the chip, there is further provided a waste fluid reservoir.

Also it is possible to form a substrate as an actuator module for fluid feeding for the purpose of mounting a portion of structural member of the micro pump in the chip. By building up this substrate further on aforesaid substrate, multi layer build-up type chip can be made. Or, an embodiment in which covering substrate representing the uppermost layer substance is made as an actuator module for feeding the fluid may be possible. In case of making a multi layer build-up type, it is necessary to select the most suitable materials for the function and a use for each substrate.

As the material of chip, various forming materials can be available in accordance with the character of each material. For instance, fluorocarbon resin, such as polystyrene, polyethylene, polypropylene, a polyvinyl chloride, polycarbonate, and polytetrafluoroethylene, polysiloxane based polymers, such as poly dimethyl siloxane, Polyolefin based polymers., such as polymethylmethacrylate, polyvinyl alcohol, and an ethylene-vinylalcohol copolymer, polyester based polymers, such as a polyethylene terephthalate and a polybutylene terephthalate, polyamide based polymers, such as 6-nylon, 6, and 6-nylon, annular cycloolefin resin, polyarylate resin, a cellulose based polymer like a cellulose acetate or a cellulose nitrate, a various inorganic matter glass, silicone, ceramics, a metal, etc. are may be listed. In particular, polysiloxane based polymers, such as polystyrene, polyethylene, polypropylene, a polyethylene terephthalate, polymethylmethacrylate, a polyvinyl chloride, polycarbonate, and poly dimethyl siloxane, silicone, and a glass especially may be preferable. However, the invention is not limited by these examples indicated.

In the micro reactor of the invention, the flow path, the element of flow path and the body are formed by resins, which are suitable for mass production, and are light in weight, robust against impact, and are easy to be disposed by incineration, so as to make the chip to be of a disposable type, which is easy to produce. The resines used are preferably desired to have good character in workability, anti-water absorbing property, chemical resistance property, heat resistance property and inexpensiveness. If the resins having these material characters in abundance as far as possible are used, number of kinds of the members constituting chip is reduced and manufacturing process can not be complicated.

For the substrate such as channel-formed substrate where flow path is formed, a resin having water repellency and hydrophobicity in which the flow path hardly distorts by absorbing water and infinitesimal amount of specimen fluid can be fed without wasting in the way is preferred. For these materials, Resin, such as polystyrene, polyethylene, polypropylene, a polyethylene terephthalate, polyethylenenaphthalate, polyethylene vinyl alcohol, polycarbonate, poly methyl pentene, fluorocarbon, and saturation annular polyolefin. Polystyrene based plastics are preferred to channel-formed substrate. Because polystyrene is superior at transparency, mechanical characters and molding character, micro work is easily applied on it. Further, polystyrene has hydrophobicity and tends to absorb protein as stated later. Using this character, by absorbing biotin-combinable protein and streptoavidin at down stream in the minute flow path, detecting section can be easily formed. Contrarily, polyethylene has large double reflection ratio and chemical resistance and heat resistance are to be considered.

Regarding heat resistance of polystyrene, a heat deforming temperature of polystyrene (18.5 kg·f·cm⁻²) is 70 to 100° C. In DNA amplification by ICAN method, as far as it is done at consistent temperature in a range of 55 to 60° C., the problem is thought not to exist. Or methyl methacrylate-styrene coplymerresin or styrene-acrylic nitryl copolymeeresin can be used. Including amplification by PCR method, in case it is needed to be heated up to around 100° C. due to the requirement of analysis, the material needs to be replaced with a material superior in heat resistance. For such materials, plastics such as polycarbonate, polyimide, polyether imide, poly Benz imidazole, polyetheretherketoneare named as examples.

To promote reaction of alanyte, often a predetermined portion of a flow path or a reaction portion in micro reactor is heated up to a predetermined temperature. In the heating area, the temperature of spot heating is usually up to around 100° C. On the other hand, in the case of a specimen that becomes unstable at high temperature, the reagent is forced to be cooled. Considering such rise and fall of the temperature of a local area in the chip, a material of adequate thermal conductivity is selected preferably. It is preferred that heating area and at least a part of non heating area adjoining the heating are formed by a material having thermal conductivity of not more than 10 W/m·K. For such materials, resin material and glass are given. By forming these areas with a material having a small thermal conductivity, spreading of heat on the surface is controlled and solely the heating area can be selectively heated.

To detect fluorescent matters or products of color reaction optically, the detecting portion which covers at lease the detecting section of the minute flow path on the micro reactor surface needs to be a member with light transmissive state. Therefore, for the light transmissive covering substrate, transparent materials as alkali glass, quartz glass and plastics can be used. In particular, as transparent plastics, polystyrene, polyethylene, polypropylene, a polyethylene terephthalate, polyethylenenaphthalate, polymethylmethacrylate, polyethylene vinyl alcohol, acryl resin, polyvinyl resin, epoxy resin, a polyvinyl chloride, a unsaturated polyester resin, polyamide resin, polyimide resin, polysulfone resin, annular cycloolefin resin, a cellulose acetate, cellulose nitrate, fluorocarbon resin, polycarbonate, or poly dimethyl siloxane may be used preferably. The covering substrate as a transparent substrate, is adhered on the channel-formed substrate so that it is formed to cover at least these structural section, the flow path and detecting section.

In case the plasticity is required, besides the light transmittance character materials such as polystyrene, polyethylene, polypropylene, a polyethylene terephthalate, poly dimethyl siloxane may be preferred. Also, in case the fluorescent light is measured as a detecting method, the fluorescent light of plastic substrate is a problem as the noise. A resin substrate to solve such noise was suggested (Unexamined Japanese Patent Application Publication No. 2003-130874). The low fluorescent material as straight-chain polyorefine, circular polyorefine and low fluorine plastic may be used.

Basic Flow Path Element

In the micro reactor of the invention, the channel-formed substrate and the covering substrate are used to from the structural sections of the pump connection section, the valve seat section and the fluid reservoir. In integrated type chip where a complicated fine work is applied on the substrate and which is manufactured by combining plurality of substrates where various kinds of flow path elements are allocated, complication of system, deterioration of the accuracy, and an increase of manufacturing cost tend to be caused. In the micro reactor of the invention, simplifying of chip forming is planned in order to realize disposable type.

Fluid Reservoir Section

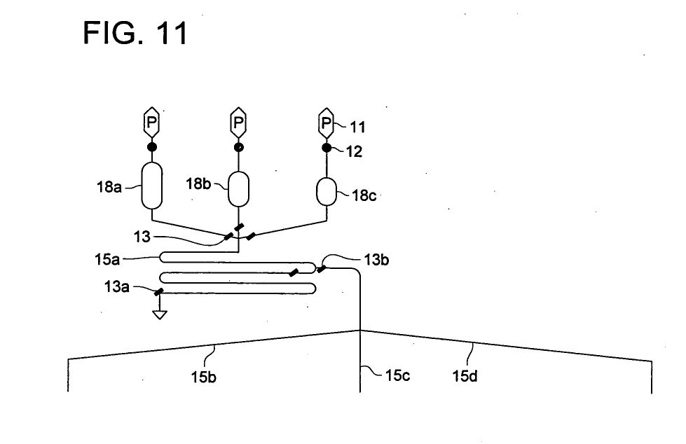

In the micro reactor of the invention, fluid reservoir section includes a specimen containing section, a reagent containing section, a control containing section, a probe containing section and a waste fluid reservoir section (FIG. 5). In addition to the fluid containing sections, for the effective mixing of fluid, reservoirs for mixing are sometimes be provided somewhere in the flow path in case of need (FIG. 11).

As for fluid containing section, recessions are formed at predetermined positions on channel-formed substrate, and they are communicated with minute flow paths, and covering substrate is spread on them thus the structural sections are formed. As such recessions, for instance, concave structures each measuring 100-500 μm in diameter and 100-500 μm in depth are formed on the fluid containing section, except for waste fluid reservoir section. The fluid containing section may be coated to prevent nonspecific adhering of biological material particularly protein, if necessary.

Minute Flow Path

The minute flow path of the micro reactor is formed on the substrate in accordance with allocation of the flow path designed in advance for the purpose (FIG. 5). The minute flow path is formed to have width of several scores to several hundreds μm preferably 50 to 100 μm, a depth of 25 to 200 μm, preferably 50 to 100 μm. If the width of flow path is less than 50 μm, flow path resistance increases and it is inconvenient for fluid feeding and detection. In the flow path with a width exceeding 500 μm, the merits of the micro scale space is reduced. The forming method is based on existing micro work technologies. Typically, transferring of micro structure using photosensitive resin through photolithography technology is preferred. Using the transfer structure, elimination of unnecessary part, adding of necessary parts and transferring of shape are carried out. After making a pattern, which forms the constructive elements of the chip by photolithography technology, the pattern is transformed onto plastic. Therefore, for the material of basic substrate, which forms the minute flow path of the micro reactor, a resin that can transfer sub-micron structure accurately and is superior in mechanical character is preferably used. Polystyrene and polydimethylsiloxane are superior in shape transferring. Injection molding and extrusion can be utilized.

In a micro space, the viscosity of the fluid is raised by the effect of capillary tube force and the flow path resistance. The material of the flow path also affects the flow path resistance of the fluid that flows in the minute flow path in this circumstance. Therefore, the hydrophobic flow path wall does not cause a rise of flow path resistance since the hydrophobic flow path wall has less interaction than the hydrophilic one. In addition, it is preferable for controlling fluid motion to stop or slow the flow. Thus, if water repelling resin is used for the substrate forming the micro flow pass, coating on the inside of the flow path is not particularly necessary. If particularly necessary, fluorine based polymer coating may be applied (for example, Unexamined Japanese Patent Document 2004-75780). Contrarily, if hydrophobic resin is used, enzymes in the reagent are absorbed in the bottom surface or the side surface of the flow path before reaching the reaction section resulting in a considerable loss. Or, there is a possibility that an impurity protein brought by the specimen is adsorbed and remains in the minute flow path to disturb layer streams or to narrow the flow path. To prevent nonspecific adsorbing of protein and DNA, inner surface of minute flow path may be coated in advance by protein as BAS(cow serum albumin) or lysophospholipid polymer. Polystyrene is particularly hydrophobic and has strong tendency to adsorb protein. Using this character, when a detecting section is formed at a downstream point on the minute flow path by adsorbing biotin-combinable protein such as streptoavidin, it is preferred that inside of the flow path up to the detecting section is coated by blocking agent in advance.

Pump Connection Section

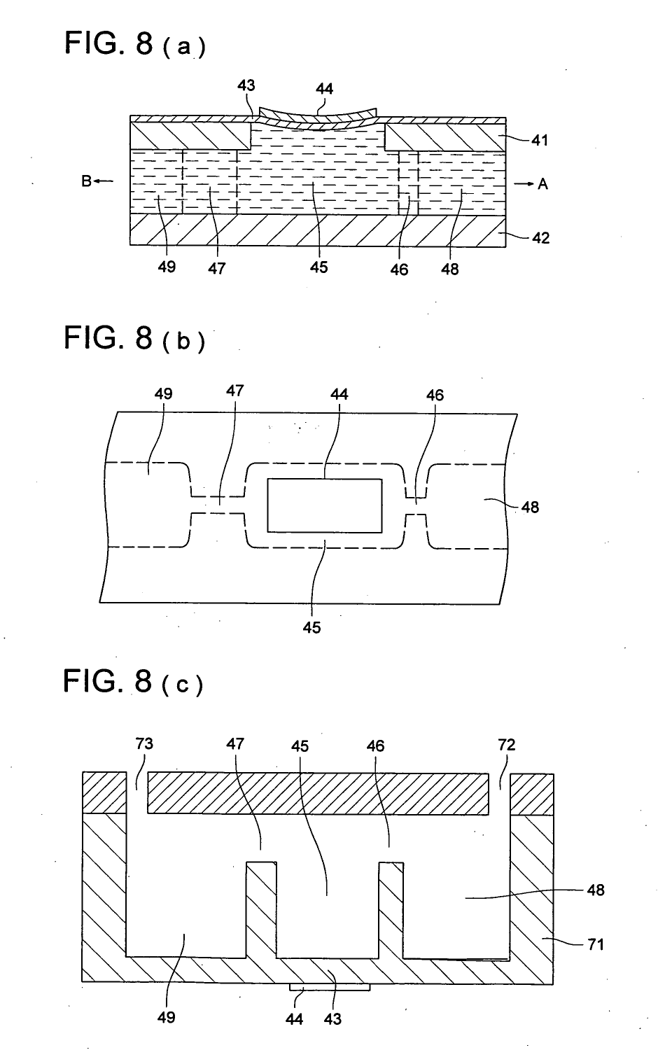

In the biological material inspection device of the invention, for each of specimen reservoir, reagent reservoir and control reservoir of micro reactor, a micro pump which feeds the fluid in the reservoirs is provided. In the invention, a piezo pump is preferably used as a micro pump (FIG. 8). Basically, fluid feeding operation section and driving section which are main body of the micro pump are incorporated in a device main body separated from micro reactor. When the micro reactor is attached to the device main body, the micro pump is connected to the micro reactor through a micro pump connection section. In this structure, at the position of the pump connecting section on the covering substrate of the micro reactor, a fluid tight connection parts, which fit with the substrate, are provided, thereby the micro pump connecting section as a inlet port to receive the fluid from the pump on device main body side is formed.

As an other embodiment, micro pump fluid feeding operation section can be provided on the half way of the minute flow path of micro reactor, and another embodiment in which its vibrating plate (FIG. 8, parts 43) is driven from main body side to function is feasible. Or, a part of micro pump structure may be incorporated in the chip by forming a substrate for actuator module for fluid feeding. The materials suitable for such substrate are required to be superior in plasticity and durability for wearing. Further it is preferred to be superior in workability of forming. Thus, glass and resin are used. In such embodiments, micro pump connecting section 12 serves as is an outlet to emit the fluid discharged by the pump to the minute flow path as a micro pump port.

VALVE

In the micro reactor of the invention, a number of backward flow preventing sections are provided at the flow path to prevent the backward flow of fluid. This backward flow preventing section is composed of a check valve in which the valve body is caused by backward flow pressure to close an opening, or of an active valve in which a valve body distorting means presses the valve body to the flow path opening section to close the opening section. A valve base of valve representing said flow path element is constructed by aforesaid substrates (channel-formed substrate and covering substrate), preferably by resin substrate with plasticity, and a valve mechanism is formed by combining its complementary parts.

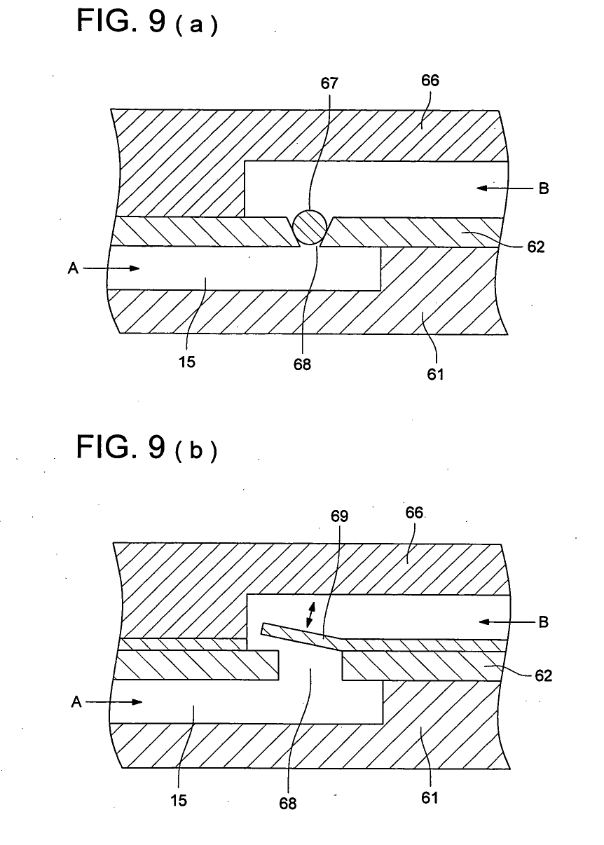

FIG. 9 is a cross sectional view showing an example of a check valve. The valve base is channel-formed substrate 61 and covering base 66, and complementary parts corresponding to these are intermediate substrate 62, micro ball 67 and resin substrate 69. In the check valve in FIG. 9(a), a passage of fluid is allowed or stopped by opening or closing opening 68 formed on intermediate substrate 62 by the movement of micro ball 67 which is a valve body. Therefore, when the fluid is fed from direction A, the passage of the fluid is allowed since the micro ball 67 is moved away from substrate 62 and opening 68 is opened. On the other hand, when the fluid flows backward from direction B, the passage of the fluid is stopped since micro ball 67 sits on substrate 62 and opening 68 is closed.

In the check valve in FIG. 9(b), plastic substrate 69, which is layered on intermediate substrate 62, and is extended in terms of its end portion, to above opening 68 moves vertically above opening 68 with the pressure of the fluid to open and close the opening 68. Thereby, when the fluid is fed from the direction A, passage of the fluid is allowed since an end of resin substrate 69 is moved away from substrate 62 by the pressure of the fluid to open opening 68. On the other hand, when the fluid flows backward from direction B, the passage of the fluid is stopped since resin substrate 69 sticks on aforesaid substrate 62 to close opening 68.

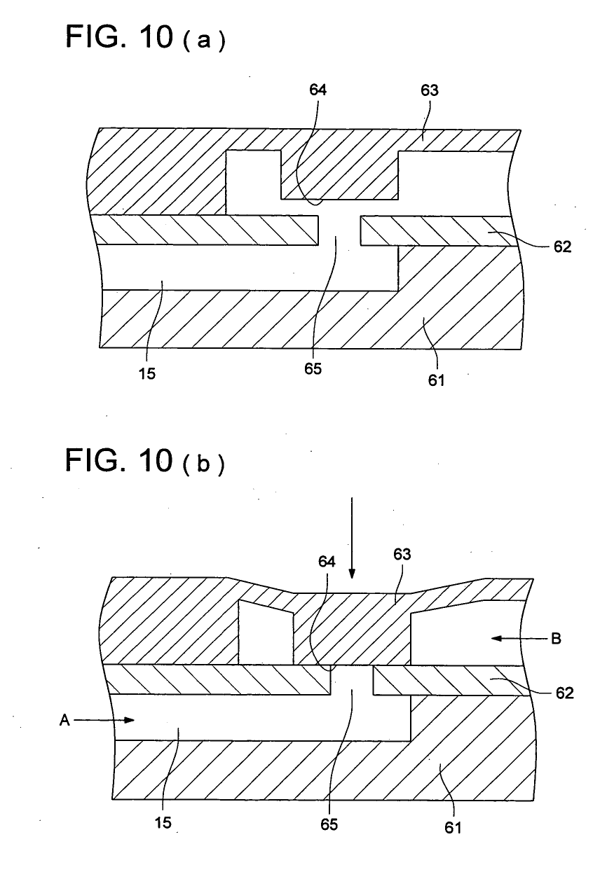

FIG. 10 is a cross-sectional view showing an example of an active valve used for flow path of micro reactor of the invention. FIG. 10(a) shows a status of open valve and FIG. 10(b) shows a status of closed valve. In this active valve, valve base is channel-formed substrate 61 and flexible covering substrate 63, and a complementary parts corresponding is intermediate substrate 62. Flexible covering substrate 63 has valve section 64 projected downward, and is layered on intermediate substrate 62 which forms opening 65.

When the valve is closed, as FIG. 10(b) shows, by pressing flexible covering substrate 63 from above portion through the means of valve distortion means such as an air pressure, a hydraulic pressure, a hydraulic piston, a piezoelectric pressure actuator and a shape memory alloy, valve section 64 adheres onto intermediate substrate 62 to cover opening 65. Thereby, it stops backward flow to direction B. The valve may also be the one having the structure which intercepts flow path by deforming the valve body itself without being limited to the one which is operated by an external driving device.

Out Line of a Biological Material Inspection Device

A micro reactor for biological material inspection of the invention has a specimen containing section, a reagent containing section, a preliminary treatment section of the specimen, a waste fluid reservoir section, a micro pump port and a minute flow path.

The minute flow path is the minute flow path branched into at least two or more paths through a micro pump and a feeding fluid dividing means, aforesaid specimen fluid after condensing is fed to downstream point in each branched minute flow path and/or a reagent encapsulated or its mixture fluid are fed to downstream points in each branched minute flow path. In the flow path constructing reacting section and in detecting section at downstream points of each branched minute flow path, a plurality of items and/or controls can be measured simultaneously.

FIG. 4 is a schematic drawing of an embodiment of biological material inspection device (which is also called “biological material inspection apparatus”) constructed from a micro reactor for biological material inspection which is detachable from a device main body, and a device main body. FIG. 5 is a schematic drawing of aforesaid micro reactor in an embodiment of the invention. The invention can be discretionary deformed and modified without departing form the spirit of the invention and these derivatives are to be included in the invention. Therefore, as for the whole or a part of the micro reactor and inspection device of the invention, structure, configuration, arrangement, shapes and forms, dimensions, materials, principle and method can be varied as far as it accords with the spirit of the invention.

FIG. 5 shows an example of typical configuration of the flow path of micro reactor of the invention. In the arrangement of the flow path and the fluid feeding element of FIG. 5, it is constructed so that the reagent may flow basically into 3 analysis flow pass (which is the flow path from being branched into three flow paths to reaching each waste fluid reservoir section 23 and such basic minute flow path may also be called “analysis flow path” hereinafter). An analysis flow path on the left is flow path for analyzing specimen and corresponds to the analysis of first item in FIG. 5. An analysis flow path in the middle is a flow path for positive control and analysis flow path on the right is a flow path for negative control. Though the number of the flow path for specimen analysis is one in FIG. 5, at least two or more of analysis flow paths need to be formed for multi item analysis. The number of analysis flow paths is limited: not only by the number of analysis items but by the chip size, and the number of the elements allocated.

The biological material inspection device of the invention has therein device main body 2 in which a micro pump, a control device to control the micro pump, a temperature control device to control the temperature and a detecting device are integrated, and micro reactor 1 attachable to the device main body 2. If a specimen fluid is injected into the specimen containing section of micro reactor 1 in which the reagent is contained in advance, and the micro reactor is attached to biological material inspection device main body 2, mechanical connection to operate the fluid feeding pump and, if necessary, electrical connection for controlling are made. Therefore, if the main body is connected with aforesaid micro reactor, the flow path in micro reactor becomes operation status. Preferably, when the measuring of a biological material is started, feeding of the specimen and the reagent, gene amplification based on mixing, reaction such as binding of analyte and probe, and detection and optical measuring of reactors are carried out automatically as a series of continuous process, and the measurement data is stored in the file together with necessary conditions and notes, then the measuring of biological material is carried out automatically.

Aforesaid detecting device is a device in which the detecting sections in the analysis flow path of each inspection items is irradiated with measuring light from, for example LED, and transmitted light or reflected light is detected through an optical detecting means such as light sensitivity of multiplier tube and photo diode. As the optical detecting means, though various kinds of optical devices, which have different principles, are available, ultraviolet/visible light spectrophotometer is preferred. The device may be one mounted on the aforesaid inspection device or the separate device connected to the inspection device when it is used. Preferably, a biological material inspection device of the invention has an integrated structure where a detecting device that optically detects the biological material contained in specimen is incorporated together with a fluid feeding means including aforesaid micro pump, a control device for micro pump and a temperature control device.

A unit that takes charge of the control system concerning each controls of fluid feeding, temperature and reaction, optical detection, and data collection and processing, constitutes main body of biological material inspection device together with the micro pump and the optical device. This main body of device can be used compatibly to specimen sample by attaching aforesaid chip. Fluid feeding order, amount and timing of reaction and detection such as aforesaid amplification, are installed as the predetermined conditions together with the control of the micro pump and the temperature and the processing of data of optical detection in a form of program in the software loaded on the biological material inspection device. In case of conventional analysis chip, when different analysis or elaboration is carried out, the micro fluid device corresponding to the contents of change has to be constructed each time. Different from this, in the invention, only aforesaid chip detachable is needed to be replaced. In case the change of control of each element is needed, only the control program stored in the main body of device has only to be modified properly.

The biological material inspection device in the invention is superior in handling and operation as it is not restricted in terms of place and time, because each component is miniaturized to be convenient in shape for carrying. It can be utilized for home treatment personally and emergency treatment since it can measure rapidly regardless of place and time. Because a number of micro pump units used for fluid feeding are mounted in main body side of the device, the chip can be used as a disposal type.

After carrying out preliminary treatment in the chip, after injecting specimen such as blood into specimen storage section of a micro reactor, by attaching the micro reactor to device main body, it is constructed to automatically carry out the predetermined reaction (for instance, in case of inspection of gene, gene amplification reaction) and process needed for its detection, and to inspect analyte simultaneously for multi item and in a short period of time. Or, the procedure in which after the micro reactor is attached to the device main body, the preliminary treatment for specimen is carried out, may be possible.

A micro reactor and a biological material inspection device of the invention are preferably used particularly for the inspection of gene or nucleic acid. The specification below is explained, quoting these gene inspection as examples. In this case, a mechanism for PCR amplification will be equipped on micro reactor. However, besides gene, basic structure (of micro reactor) for other biological material is almost the same. Usually, it is only needed to change preliminary treatment section for specimen, the reagents and the probes, and allocation and number of fluid feeding element may vary in that case. For instance, it is possible for those skilled in the art to change a type of analysis easily by applying modification including minor alteration of flow path element and by placing reagent necessary for immunoassay method on micro reactor. The biological material other than gene herein is various metabolism matters, hormone and protein (including enzyme, antigen and so forth).

Specimen

The specimen to be measured in the invention is a sample containing an alanyte originated from organism. There is no restriction in the samples itself and for example, almost all samples originated from organism such as whole blood, blood plasma, blood serum, baffy coat, urine, dejection, saliva and sputum are applicable. In case of gene inspection, the gene as a nucleic acid which is a template of amplification reaction, DNA or RNA is the alanyte. The specimen may be the one isolated or prepared from samples possibly contain such nucleic acid. Therefore, besides the above samples, cultured cell substance; the samples contain nucleic acid such as virus, bacteria, yeast, the samples contain nucleic acid such as plant and animal; the samples possibly contain or entrain germs, and other samples which may contain nucleic acid are applicable.

Conventional technologies can be used for the method preparing gene, DNA or RNA from such samples without specific restriction.

In the micro reactor of the invention, comparing with manual operation using conventional devices, the required amount of specimen is extremely small. For instance, only about 2 to 3μ litter of blood is injected to a chip measuring several centimeter in length and in width. For instance, in case of a gene, 0.001 to 100 ng of DNA is to be injected.

Specimen Containing Section

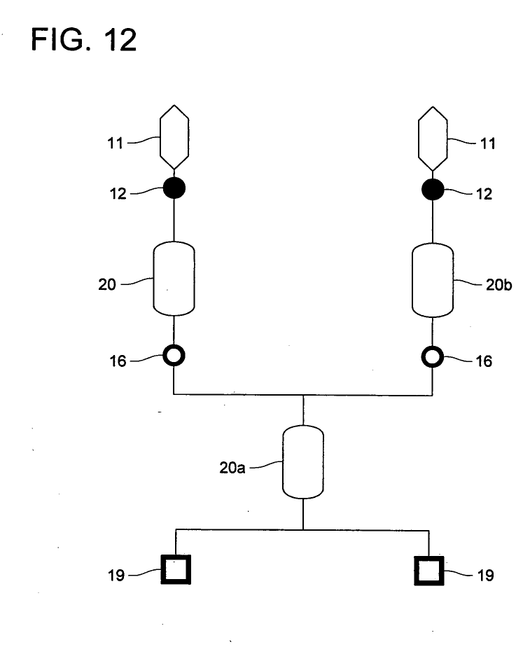

Specimen containing section 20 of a micro reactor of the invention has a structure shown by FIG. 6 and FIG. 12. The specimen containing section 20 communicated to specimen injection section stores the specimen temporarily and feeds the specimen to mixing section. The specimen injected to specimen storage section 20, which is connected to micro pump 11 and pump connecting section 12, by operation of them, is fed to specimen preliminary treatment section 20a. In specimen preliminary treatment section 20a, the specimen is treated by treatment fluid fed from specimen treatment fluid containing section 20b. Such specimen preliminary treatment sections 20a are allocated according to requirement. A preferred specimen preliminary treatment includes separating or condensing of alanyte, and removing of protein. Therefore, specimen preliminary treatment section 20a may include a separation filter, a resin for adhesion and beads.

Specimen Preliminary Treatment Section

Generally, it is normal practice that an biological sample such as blood or urine needs to have a preliminary treatment of the specimen prior to analysis to remove unnecessary components (protein and ionic substance) contained in the sample. The preliminary treatment differs with the kind of specimen and the method of analysis used. Usually, in case of the preliminary treatment for the biological sample frequently used, the treatments such as cytoclasis (bacteriolysis or cythemolysis) or solubilization, extraction, deproteinzation, condensing, adsorbing, desorption, washing, dialysis (desalting), filteration, hydrolysis or derivatization are performed. For instance, to prevent clogging of the minute flow path, insoluble impurities have to be removed. Prior to detection, it is preferable to condense and isolate objective matters in advance. Depending on the specimen, concentration of matter to be detected is extremely low. In this case, since the amount of specimen injected is limited (several μ liters for a chip of several centimeters square) it cannot fall within a measurable range as it is. Therefore, a preliminary manipulation of condensing or isolating of the objective substance is needed. Further, in case the specimen fluid is viscous, it may be diluted to adjust the viscosity or the interfacial tension as occasions demands, in order that the fluid is fed through the minute flow path in a form of streamlined flow smoothly. The viscosity (measured by Ostwald process) of the fluid flowing through the minute flow path is to be adjusted to be 15 mPa·s or less, preferably 10 mPa·s or less at 37° C.

If preliminary treatment of specimen can be carried out safely in the same chip where analysis and detection are carried out, and if the specimen capable of being measured can be prepared rapidly and automatically, building such embodiments of chip is very significant.

The method of preliminary treatment usually differs with cases, according to the kind of sample, the kind of objective substance, existence concentration, existence of interfering substance. Therefore, in the micro reactor of the invention, preliminary treatment section 20a to carry out preliminary treatment in case of need is provided from the view point of specimen and analysis. In specimen treatment fluid container 20b communicated to the preliminary treatment section 20a, bacteriolysis reagent, cythmolysis reagent, extraction fluid, denaturation fluid, washing fluid and eluant are encapsulated.

Preliminary Treatment Means

In the micro reactor of the invention, though aforesaid preliminary treatment does not specify its embodiment of a carrier that selectively adsorbs biological materials, germs or viruses, it is specifically gel or membrane such as filter, bead and agarose. In accordance with purposes, the carrier may be a combination of a plurality of filters or combination of aforesaid carriers. For availability and usability, a filter is preferred and a preliminary treatment means having layered filters is preferred.

A filter that traps DNA is quoted as a filter adsorbs aforesaid biological material. A filter trapping DNA may be, for instance, a filter that adsorbs DNA molecule specifically under some conditions. The mesh of the filter is to be considered for sizes of germs or viruses. Shape and thickness of the filter layer are determined in accordance with purposes. For instance, to filter and remove insoluble matters and dust first and carry out a process afterward, two kinds of filters having different sizes may be used together. Shape of the filter is discretionary such as a configuration of piled layers, a configuration of filled up particles, a layer of resin and a configuration of congregated hollow strands.

Then the specimen after the preliminary treatment is separated into not less than two minute flow paths for analysis by aforesaid feeding fluid dividing means and fed to subsequent analysis flow path that is communicated at a downstream side. The divided specimen flows from sample port 19 shown by FIG. 6 and FIG. 12 into a minute flow path where reagent is flowing to be merged. In this case, a port from where the specimen runs out and an analysis flow pass to be merged with the port and to be intersected at a certain place on a basis of two-level crossing by separating feeding fluid in order that the specimen may flow through than two analysis flow paths to merge with reagents. The reason for the above positional relation is as follows:

As FIG. 6 shows, elements such as specimen containing section 20 and specimen preliminary treatment section 20a are preferably allocated to be closer to the downstream than reagent storage section 18 is in analysis flow path (micro flow pas on the left) for specimen analysis as shown in FIG. 5. Therefore, in FIG. 5, in case the measuring item of the specimen is one, containing section 20 and one specimen reservoir section 17b illustrated are enough. On the other hand, in case the measuring items are not less than two, the specimen needs to be divided in accordance with the number of the items as mentioned above and needs to be fed to each analysis flow path. Thus, aforesaid elements are allocated at appropriated positions (they do not have to be right above) on plural analysis flow paths. The positional relation is also shown in FIG. 6 as an example. Before the specimen and the reagent are merged, a flow path where the specimen flows from port 19 has to intersect a flow path where the reagent flows on a basis of two-level crossing. Meanwhile, as illustrated, when specimen preliminary treatment section 20a is installed as well, it is convenient that specimen preliminary treatment section 20a is located to be lower than specimen containing section 20 to dispose unnecessary fluid into waste fluid reservoir section 23.

Meanwhile, at a portion on the upper surface of the specimen containing section where specimen is injected (specimen injecting section), it is preferred that a plug made from elastic material such as rubber material is formed on the covering substrate or the specimen injecting section is covered by a plastic reinforced film such as poly dimethyl siloxane (PDMS), in order to prevent leakage of the specimen to outside, infection and pollution, and to keep sealing performance. For example, the specimen in a syringe is injected through a needle piercing aforesaid rubber plug or a needle penetrating through a pinhole on a lid. In case of the former, it is preferred that the hole closes right after the needle is withdrawn. Or, other specimen injecting mechanisms may be employed.

Further, for example when DNA is extracted, AGPC method is common. The materials forming the specimen containing section and the specimen preliminary treatment section are preferably durable material against organic solvent and acid.

Waste Fluid Reservoir Section

The micro reactor of the invention has waste fluid reservoir section 23 to capture the waste fluid resulted from condensing and measuring of the specimen, and that reservoir is formed by further adhering a substrate onto the bottom of channel formed substrate as a bottom substrate so as to cover a concave portion formed on the bottom of the channel formed substrate (FIG. 7). Therefore, the waste fluid reservoir section provided at the bottom of the micro reactor is a sealed waste fluid reservoir to reserve all of excessive specimen, washing fluid and waste fluid and waste fluid produced through the process of isolation and condensing of specimen, and waste fluid resulted from measuring and reaction of specimen. For such waste fluid, it is less troublesome to reserve the waste fluid inside automatically rather than to eject it outside the micro reactor for processing. Preferably, at least, the reservoir is sealed structure that is communicated with aforesaid specimen preliminary treatment section, the reaction section at the flow path, and the end of the detecting section, and has necessary capacity, namely, it is a cavity where penetrating holds communicating with respective sections which produce waste fluid are formed. Waste fluid reservoir section 23 may be a single chamber or a configuration of multi ward cavity divided by a plurality of wards. Its capacity and shape are not restricted in particular. Meanwhile, the mother material constituting the waste fluid reservoir section is preferably a material endurable to organic solvent and acid.

Reagent Containing Section

In the micro reactor of the invention, predetermined amount of necessary regent is encapsulated in reagent containing section 18 of the micro reactor in advance. The micro reactor of the invention does not require a necessary amount of the reagent to be charged, each time of use and is ready for immediate use. In the preferred embodiment of the invention, a micro pump is connected to upstream point of the reagent containing section, and by supplying a drive fluid to the reagent containing section side through the micro pump, the reagent is extruded to the flow path and fed.

In case biological material in the specimen is analyzed, the reagent necessary for the measuring is usually known publicly. For example, when an antigen existing in specimen is analyzed, a reagent containing an antibody correspond to it or preferably monoclonal antibody is used. The antibody is labeled with biotin and FITC. In the reagents for gene inspection, preliminary treatment reagent used in aforesaid specimen preliminary treatment may also be included together with various reagents for gene amplification, probes used for detection, and coloring reagents, if necessary.

Micro Pump and Pump Connecting Section

In the present embodiment of the invention, micro pump 11 to feed the fluid contained is provided on each of containing section of specimen containing section 20, reagent containing section 18, positive control containing section 21h and negative control containing section 21i. Micro pump 11 is connected to upstream point of regent containing section 18. Through micro pump 11, the drive fluid is supplied to the reagent containing section in order to extrude and feed reagent to the flow path. A micro pump unit is mounted on the device main body (biological material inspection device) which is separated from micro reactor and connected to the micro reactor through pump connecting section 12 by attaching the micro reactor onto the device main body.

In the present embodiment of the invention, a piezo pump is used as the micro pump (FIG. 8). That is the micro pump is the piezo pump provided with a first flow path whose flow resistance varies with the change of pressure difference, a second flow path in which a ratio of the change of flow resistance for the change of the pressure difference is smaller than that of the first flow path, a pressure chamber connected to both the first flow path and the second flow path, and an actuator which changes the pressure inside aforesaid pressure chamber. The details are mentioned in aforesaid Patent Document 1 and 2.

Feeding Fluid Dividing Means

In the invention, in case multi items are analyzed for one specimen, and positive control and negative control are analyzed at the same time, each of the reagent and specimen needs to be divided into two or more and fed to each analysis flow path. The feeding fluid dividing means is provided for it. Practically, as FIG. 5 and. FIG. 11 show, the feeding fluid dividing means is constituted of branched minute flow paths, feeding fluid control section 13 and backward flow preventing section 16.

Feeding fluid control section 13 stops passage of the fluid until the fluid feeding pressure in the positive direction reaches a predetermined pressure, and allows passage of the fluid by applying fluid feeding pressure exceeding the predetermined pressure. Also backward flow preventing section 16 preventing backward flow of fluid in the flow path is constituted of a check valve in which a valve body closes a flow path opening through backward flow pressure or an active valve in which a valve body is pressed onto a flow path opening through a valve body distorting means to close the opening.

In the minute flow path of the micro reactor of the invention, the feeding of fluid in branched flow path, the fixed amount of feeding fluid and mixture of each fluid are controlled by a aforesaid micro pump, a feeding fluid control section which is able to control passage of fluid through the pump pressure of aforesaid micro pump and a backward flow preventing section which prevents backward flow of the fluid in the flow path. Through the fluid dividing means and operation of micro pump 11, the reagent and the specimen are divided at adequate ratio.

Reacting Section

In the structure of the reaction section, a specimen containing section which contains aforesaid specimen and a reagent containing section which contains reagent are provided in each flow path at the upstream point of a merging section which merges a solution of specimen including a biological material (alanyte) to be measured and a reagent (mixed fluid). There are provided pump connection sections at upstream position of each containing sections, and aforesaid micro pumps are connected to the connecting sections to supply driving fluid from each micro pump. Thus necessary reaction for analysis such as a gene amplification reaction, the trapping of analyte or an antigen-antibody complex reaction are started, by extruding and merging aforesaid specimen fluid in aforesaid each containing section and aforesaid reagents. The mixing of reagent and reagent, and the mixing of specimen and reagent can be done in a single mixing section at a desired mixing rate. Or, the mixture can be done by dividing either one or both of them, to provide a plurality of mixing sections to mix at a desired mixing rate eventually. The embodiments of such reacting section are not particularly limited and various embodiments can be designed. For example, if detecting material is immobilized on the beads, by combining beads and the micro reactor, the reacting surface area can be drastically increased. As studies featuring this character, there are reported one to conduct hybridization by immobilizing DNA probe on beads, to introduce the DNA probe into the minute flow path of the micro reactor chip, and one in which dramatic speedup of immunologic tests is realized by introducing the beads into the reacting section of the micro reactor to carry out antigen-antibody complex reaction.

Basically, it is preferred to provide a merging section in which at least two types of fluids including a reacting reagent are fed by the micro pump and merged, a minute flow path located beyond aforesaid merging section in which aforesaid respective fluids are diffusively mixed, and a fluid containing section, located beyond the end of downstream point of aforesaid minute flow path and is constituted of wider space than the minute flow path, in which the mixture fluid, which is diffusively mixed in the minute flow path is reserved for reaction.

Method of Gene Amplification

In gene inspection which uses the micro reactor of the invention, the method of amplification is not limited. For example, in DNA amplification technology, PCR amplification method, which is popularly used in many fields, can be used. Various conditions to conduct this amplification technology, are being studied in detail and the studies including improvements are published in various documents.

ICAN (Isothermal chimera primer initiated nucleic acid amplification) method recently developed as an improvement of PCR has a characteristic to conduct DNA amplification under a certain operational temperature between 50 and 65° C. in a short period of time (Patent No. 3433929). Therefore, in micro reactor of the represent invention, ICAN method is a preferable technology because of simple temperature management.

Detecting Section

In the micro reactor of the invention, a detecting section for detecting analyte, for instance, amplified gene, is located at downstream point of the reacting section of the minute flow path. To make optical measuring possible, at least a detecting portion is made of light transmissive material, preferably light transmissive plastic. Further, biotin affinity protein (avidin+, streptoavidin+, extraavidin(R), preferably streptoavidin) adsorbed on the detecting section in the minute flow path is bound peculiarly with biotin labeled by a probe material or biotin labeled by 5′ end of a primer used for gene amplification reaction. Thereby, the probe labeled by biotin or the amplified gene is trapped by the present detecting section.

The detecting method of separated alnalyte or DNA of amplified target gene is not limited however, as a preferred embodiment, basically it is conducted in the following procedure. That is, using aforesaid micro reactor, (1a) a specimen or DNA extracted from a specimen, a specimen, cDNA synthesized by reverse transcript reaction from RNA extracted from specimen or a primer which is modified by biotin at 5′ position are fed from their containing sections to minute flow path downstream. After conducting a process of amplify gene in minute flow path of reacting section, a process in which amplified gene is denaturalized to single-strand, by mixing amplified reaction fluid including amplified gene in the minute flow path with alteration fluid, and a process in which aforesaid amplified gene is trapped, by feeding treatment fluid which is made by denaturalizing amplified gene to single-strand to detecting section in micro flow pass where biotin affinity protein is adsorbed, DNA probe, which end is fluorescently labeled by FITC (fluorescein isothiocyanate), is allow to flow in the detecting section where amplified gene is trapped and is immobilized onto the gene to complete hybridization. (The one in which an amplified gene and a fluorescently labeled probe DNA are hybridized in advance may be trapped in detecting section). (1b) A peculiar antibody corresponding to the alanyte such as, an antigen existing in specimen, metabolite, hormone or preferably a reagent including monocronarl antibody is mixed with a specimen. In this case, the antibody is labeled by FITC. Therefore, a product obtained by antigen-antibody reaction includes biotin and FITC. This is fed it to the detecting section in the minute flow path where biotin affinity protein (preferably streptoavidin) is adsorbed, and it is immobilized on the detecting section through the binding of biotin affinity protein and biotin. (2) A gold colloid solution whose surface is modified by anti FITC antibody which is specifically bound with FITC is made to follow through aforesaid minute flow path, so that the gold colloid solution is adsorbed by immobilized alanyte and FITC of anti body reactant or by FITC modified probe hybridized to gene. (3) The concentration of gold colloid in aforesaid minute flow path is optically measured.

When streptoavidin is immobilized in the minute flow path formed on a polystyrene substrate, specific chemical treatment is not necessary. Simply applying biotin affinity protein has only to be applied in minute flow path at downstream position of amplification reaction section, so that biotin affinity protein may be adsorbed in the flow path. The probe is to be bound with alanyte, and in case substance to be measured is protein alanyte, a specific antibody which is bound with FITC, a fluorescent label for detection, and aforesaid biotin, corresponds to the probe. Also, As a DNA probe for generic inspection, fluorescently labeled origodeokishinucreotid is used preferably. For the DNA base sequence, a based sequence complementary with a part of basic sequence of the target gene for detection is selected. By selecting a base sequence of probe DNA appropriately, the target gene is bound specifically and high sensitive detection is possible without being interfered by coexisting DNA and background.

As fluorescent dye labeling a probe, fluorescent material in the public domain such as FITC, RITC, NBD, Cy3, Cy5 can be used. FITC is particularly preferred since it can obtain anti FITC antibody, for instance, gold colloid anti FITC anti mouse IgG. Instead of fluorescent dye, gegokishigenin (DIG) can be used for the label of probe DNA. In this case, anti DIG-alcarihosfatarzelabeling antibody is used as a replacement of anti FITC antibody.

Though measuring fluorescent of fluorescent dye FITC is possible, photo fading of fluorescent dye and background noise are necessary to be considered. Eventually, the method enabling high sensitive measuring through visible light is preferred. Absorption spectrophotometry through visible light is superior, because the equipment used is more compatible than that of fluorescent photometry, interfering factor is fewer and data processing is easier. Instead of using optical detection of gold colloid anti FITC anti mouse IgG, aforesaid probe can be labeled by horseradish parokishidarze (HRP) in place of aforesaid fluorescent dye. For detection, calorimetric reaction catalyzed by this enzyme can be used. For this purpose, typical calorimetric material such as 3,3′,5,5′-tetorametilbengegen(TMB)+, 3,3′-geaminobengegen(DAB)+, p-fenirengeamin(OPD) are known. Other than these, calorimetric series enzyme such as alcarihosfatarze+, garactoshidarze also can be used.

Measuring of Control

In analysis of biological material, it is carried out in parallel with analysis of specimen by adding usually negative control to the analysis, because it is necessary for correcting contamination, for example, coloration and fluorescence of substance intermingled in reagents. Further, to improve reliability of the result of analysis, it is necessary to add positive control for quality control of analysis. It is useful for detecting interfering factors in regents to be added, and for verifying appropriateness of determined conditions and non-specific synergy effect. Also, sometime internal control sometimes needs to be added, and it is useful specifically for quantitative analysis.

It is essential to carry out positive control and internal control simultaneously, specifically for gene amplification through PCR method and antigen-antibody reaction, because checking of proper performance of PCR reaction and antigen-antibody reaction is also specifically needed. For instance, in case a trouble occurs, the foregoing is ideal to verify whether it is caused by established conditions, regents, operations, analysis systems or specimens itself or not. In particular, since PCR method can amplify a minute amount of gene existing in specimen to several hundreds of thousands times or more—several millions times or more as many as its original amount on effect of pollution such as cross contamination is remarkably serious.

Setting of these controls useful for judging false positive and false negative, follows the custom of conventional analysis technologies. In the configuration of the flow path of the micro reactor of the invention, besides an analysis flow path specimen, setting of control can be done in analysis flow path separate from specimen with the same reagent and under the same condition simultaneously.

Outline of the Micro-Reactor and Biological Substance Inspection Device

Firstly, an outline of the micro-reactor is explained. FIG. 5 is a schematic drawing of a micro-reactor for gene inspection according to one embodiment of the present invention.

The microreactor 1 shown in FIGS. 5 and 11 is made up of a chip composed of an adequate combination of the members made of a plastic resin, glass, silicon and ceramic. The minute flow path and the frame of the microreactor are preferably made of plastics characterized by easy, economical processing and molding, and easy incineration and scrapping. Of these plastics, the polystyrene resin is excellent in molderability and is very likely to adsorb streptoavidin, as will be described later. The detecting site can be easily formed on the minute flow path. In this respect, use of polyethylene is preferred. Further, for optical detection of a fluorescent substance or a color reaction product in the microreactor, at least the detecting portion, covering the detecting site of the minute flow path, on of the surface of the microreactor must be transparent or must be made of transparent plastics.

The microreactor chip for gene inspection is provided with a sample storage section, a reagent storage section, a probe DNA storage section, a control storage section, a flow path, a pump connecting section, a liquid feed control section, a backflow preventing section, a reagent determining section and a mixing section. They are installed at functionally adequate positions according to the micromachining technology. If further required, a reverse transcriptase part may be arranged. The sample storage section communicates with the sample injection section. It stores samples temperature temporarily and supplies samples to the mixing section. If required, the sample storage section can be assigned with the functions of blood cell separation and adjustment of liquid sample viscosity. Mixing between reagents, and mixing between sample and reagent can be done at a desired rate by a single mixing section. Alternatively, one of them or both can be separated and a plurality of confluence sections can be arranged so that a desired mixing ratio can be obtained in the final phase.

Such a sample as blood is injected into the aforementioned sample storage section of the microreactor and the apparatus proper is mounted on the microreactor, whereby processing required for gene amplification reaction and detection is carried out automatically in the chip, and gene inspection is conducted simultaneously for a plurality of items in a shorter time. In the preferred arrangement of the microreactor for gene inspection according to the present invention, the microreactor is filled with a predetermined amount of required reagents in advance. The microreactor is used for each sample as a chip for predetermined amplification reaction with the sample DNA and RNA and detection of the amplification product.

In the meantime, the control system to provide control of the liquid feed, temperature and reaction, and the unit in charge of optical detection, data collection and processing, together with the micro-pump and optical apparatus, constitute the biological substance inspection device proper of the present invention. This device proper can be used for the samples in common when the aforementioned chip is mounted thereon. This arrangement allows quick and efficient processing of a great number of samples. In the conventional art, when analysis or synthesis of different contents is conducted, it has been necessary to configure a micro-fluid device conforming to the contents to be modified. By contract, the present invention requires the replacement of only the replaceable chip. Modification of the control of each device element, if required, can be achieved by changing the control program stored in the apparatus proper.

Any of the components used in the gene inspection device of the present invention is downsized for easy portability, and is characterized by excellent workability and maneuverability, independently of the place and time of use. Since this device ensures quick measurement independently of the place and time of use, it can be used for emergency medical care, or for private application in the field of home medical care. The apparatus proper incorporates a large number of micro-pump units used to feed the liquid, and others, and therefore, the chip can be used as a disposable unit.

The biological substance inspection microreactor and biological substance inspection device of the present invention have been outlined with reference to gene inspection. The present invention can be embodied in a great number of variations with appropriate modification or additions, without departing from the technological spirit and scope of the invention claimed. To be more specific, all or part of the microreactor and inspection apparatus can be formed in a great number of variations, if the structure, arrangement, layout, configuration, dimensions, material, scheme and method do not depart from the technological spirit and scope of the present invention.

The gene screening microreactor of this invention comprises on a single chip:

a specimen storage section into which a specimen or DNA extracted from a sample is poured;

a reagent storage section into which the reagent used in the gene amplification reaction is stored;

a positive control storage section into which the positive control is stored;

a negative control storage section into which the negative control is stored;

a probe DNA storage section into which the probe DNA for hybridization with the gene for detection that has been amplified by a gene amplification reaction is stored;

a flow path for causing the storage sections to communicate; and

a pump connection portion which can connect with each of the storage sections and with a separate micro-pump which feeds fluid in the fluid flow path, and

after the micro pump is connected to chip via the connection portion, and the specimen or the DNA extracted from the specimen stored in the specimen storage section and the reagent stored in the reagent storing section are fed to the flow path and then mixed in the flow path to cause an amplification reaction, the processing fluid resulting from processing the reaction fluid and the probe DNA stored in the probe DNA storage section are fed, and mixed and hybridized in the flow path, and the amplification reaction detection is performed based on the reaction products, and similarly, the positive control stored in the positive control storage section and the negative control stored in the negative control storage section undergo amplification reaction with the reagent stored in the reagent storage section in the flow path, and then hybridization with the probe DNA stored in the probe DNA storage section in the flow path and amplification reaction detection is performed based on the reaction products.

The gene screening microreactor comprises a reverse transcription enzyme storage section into which the specimen or RNA extracted from the specimen stored in the specimen storage section is poured, and which stores the reverse transcription enzyme for synthesizing cDNA from the RNA stored therein using a reverse transcription reaction, and

the specimen or the RNA extracted from the specimen stored in the specimen storage section and the reverse transcription enzyme stored in the reverse transcription storage section are fed to the flow path and mixed in the flow path and cDNA is synthesized and then the amplification reaction and the detection thereof is performed.

Gene Amplification Reaction Section

An amplification of gene of specimen is conducted as follows at a predetermined position in a micro flow passage in the micro-reactor of the present invention, that is, at a gene amplification reaction section. A sample storage section for storing the aforementioned sample and a reagent storage section for storing reagent solution are arranged along the flow path upstream of the confluence section for merging the solution containing the biological substance to be measured, with the reagent (liquid mixture). At the same time, pump connecting sections are provided upstream of these storage sections. The aforementioned micro-pumps are connected to these pump connecting sections, and the drive solution is supplied from each micro-pump, whereby the sample solution and the reagent inside each storage section are pushed out and are merged. These steps initiate reaction required for the analysis such as gene amplification reaction and antigen-antibody reaction. Such an embodiment of the reaction site is not restricted thereto. The reaction site can be embodied in a great number of variations.

Basically, the reaction site preferably includes: a confluence section for allowing two or more liquids containing a reaction reagent to be fed and merged by the micro-pump; a minute flow path, arranged forward of the confluence section, for diffusing and mixing the liquids; and a liquid reservoir arranged forward of the downstream end of the minute flow path and composed of a space wider than the minute flow path, the liquid reservoir storing the liquid mixture diffused and mixed in the flow path so that the liquid mixture is subjected to reaction.

Specimen

The specimen of this invention to be determined is a gene, DNA or RNA as the nucleic acid which is the matrix for the amplification reaction in the case of gene screening. The sample may also be one prepared or isolated from a sample which may include this type of nucleic acid. The method for preparing genes, DNA or RNA from this sample is not particularly limited and known techniques may be used. In recent years, techniques for preparing genes, DNA or RNA from a living sample for gene amplification have been developed and these may be used in the form of a kit or the like.

The sample itself is not particularly limited and includes almost all samples of biological origin such as whole blood, serum, Buffy coat, urine, feces, saliva and sputum; samples including nucleic acid such as cell cultures, viruses, bacteria, mold, yeast, plants and animals; samples that may include, or into which microorganism are blended; and various other samples that may include other nucleic acids.

The DNA can be separated from the sample and purified in accordance with a usual method by phenol chloroform extraction and ethanol sedimentation. Use of a high concentration chaotropic sample such as guanidine hydrochloride and isothiocyanic chloride which is near saturation concentration for isolating nucleic acid is generally known. A method, in which the specimen is directly processed with a protein decomposition enzyme solution including a surfactant (PCR Experiment Manual by Takashi Saito, published by HBJ publishers 1991, P309), rather than using the phenol chloroform extraction described above, is simple and quick. In the case where the genome DNA or the gene obtained is large, a suitable control enzyme such as BamHI, BgLII, DraI, EcoRI, EcoRV, HindIII, PvuII and the like and performing fragmentation according to a conventional method. In this manner, DNA and aggregates of fragments thereof can be prepared.

The RNA is not particularly limited provided that the primer used in the transcription reaction can be produced. Aside from whole RNA, RNA molecule groups such as retroviral RNA which functions as a gene, mRNA or rRNA which are direct information transmission carriers for the expressed gene can be screened. These RNAs may be converted to cDNA using a suitable reverse transcription enzyme and then analyzed. The method for preparing mRNA can be done based on known technology and reverse transcription enzymes are readily available.

The quantity of sample required in the microreactor of this invention is much less than that for the operation using the device of the prior art. For example, in the case of a gene, the quantity of DNA required is 0.001 to 100 ng. As a result, there are no limitations in terms of the sample for use of the microreactor of this invention including case where only an extremely small quantity of sample can be obtained, and when the quantity is inevitably small because of the nature of the sample, and thus screening cost is reduced. The sample is introduced from the introduction section of the “specimen storage section” described above.

Amplification Method

The amplification method in the microreactor of this invention is not particularly limited. For example the DNA amplification method may be the PCR amplification method which is used extensively in a wide range of applications. The various conditions for implementing the amplification technology have been studied in detail, and are described along with modifications in various documents. In PCR amplification, temperature control in which temperature is increased and decreased between 3 temperatures is necessary, but a flow path device which is capable of favorable control of the microchip has already been proposed by the inventors of this invention (Japanese Patent Application Laid-Open 2004-108285). This system device should be used in the amplification flow path of the chip of this invention. As a result, because the heat cycle can be switched to a high speed and the minute flow path functions as a micro reaction cell having low heat volume, the DNA amplification is performed in much less time than the conventional system in which DNA amplification is performed manually using a micro tube, a micro vial or the like.

In the recently developed ICAN (isothermal chimera primer initiated nucleic acid amplification) in which the complicated temperature controls of PCR reaction is unnecessary, the DNA amplification can be carried out is a short time at a suitably selected fixed temperature which is 50° C. to 65° C. (Japanese Patent No. 3433929). Accordingly, the ICAN method is a suitable amplification technique for the microreactor of this invention because the temperature control is simple. The method which takes 1 hour for manual operation, takes 10 to 20 minutes and preferably 15 minutes to completion of analysis in the bioreactor of this invention.

Other improved PCR methods or modified PCR methods can be used for the DNA amplification reaction. The microreactor of the present invention is flexible enough to conform to any of these methods by flow path design changes. When any DNA amplification method is to be used, those skilled in the art can easily introduce that method since the details of the technique are disclosed.

Reagents

(i) Primer

The PCR primer is 2 types of complementary oligonucleotide on both ends of the DNA strand with a specific site for amplification. The settings have already been developed by dedicated applications and one skilled in the art can easily make the primer using a DNA synthesizer or a chemical synthesizer. The primers for the ICAN method are the DNA and RNA chimera primer and the preparation method for these substances have already been technologically established (Japanese Patent No. 3433929). It is important that the setting and selection of the primer is such that most suitable substance for affecting the results and efficiency of the amplification reaction is used.

In addition, if biotin is bound with the primer, the amplified DNA product can be fixed on a substrate via binding of streptavidin with the substrate and a fixed quantity of the amplification product can be supplied. Other examples of primer marker substances include digoxigenin and various fluorescent dyes.

(ii) Reagents for Amplification Reaction

The enzymes which are the reagents primarily used in the amplification reaction can be readily obtained by any of the PCR or ICAN methods.

Examples of the reagent in the PCR method include at least 2-deoxynucleotide 5′-triphosphate as well as Taq DNA polymerase, Vent DNA polymerase or Pfu DNA polymerase.

The reagents in the ICAN method include at least 2′-deoxynucleotide 5′-triphosphate, a chimera primer that can be hybridized specifically with the gene to be detected, a DNA polymerase having chain substitution activity, and the endonuclease RNase.

(iii) Control

Internal control for the marker nucleic acids (DNA, RNA) is used for amplification monitoring or as an internal standard substance when the quantity is fixed. The sequence of the internal control is such that the primer which is the same as the primer for the specimen can be amplified in the same way as the specimen in order to have a sequence that can be hybridized at both sides of the sequence which is different from the specimen. The sequence of the positive control is a specific sequence which detects the specimen and is the same as that of the specimen in the portion which the primer will hybridize. The nucleic acid used in the control (DNA and RNA) may be any described in a known documents. The negative control includes all reagents other than nucleic acids (DNA, RNA) and are used to check whether there is contamination and for background correction.

(iv) Reagent for Reverse Transcription

In the case of RNA, the reagent for reverse transcription is a reverse transcription enzyme or a reverse transcription primer for synthesizing cDNA from RNA and these are commercially available and easily obtained.

A prescribed quantity of the bases for amplification (2′-deoxynucleotide 5′-triphosphate) and the gene amplification reagent and the like respectively are sealed beforehand in the reagent storage section of one microreactor. Accordingly, when the microreactor of this invention is to be used, it is not necessary to supply the necessary quantity if reagent each time, and thus the device is ready for immediate use.

Detecting Section

The DNA detection method for the target gene that has been amplified in this invention is not particularly limited and any suitable method may be used as necessary. A visible light spectrophotometry method, a fluorophotometry method, an emitted luminescence method are considered mainstream as the suitable methods. Further examples include an electrochemical method, surface plasmon resonance, and quartz oscillator microbalance and the like.

In the gene inspection microreactor of the present invention, a detection site for detecting the amplified gene is arranged downstream of the gene amplification reaction site of the flow path. At least the detection portion of the microreactor is transparent or is preferably made of transparent plastic to permit optical measurement. Further, the biotinophilic protein for trapping the amplified gene is adsorbed on the detection site of the minute flow path. The flow path of the detection site is preferably made of plastic as well. Alternatively, at least the minute flow path is preferably made of polystyrene.

In the detection site of the present microreactor, presence or absence of amplification reaction is checked with respect to the hybridized product resulting from hybridization between the amplified gene and probe DNA. Specific combination with the target gene is ensured by adequate selection of the base sequence of the probe DNA, and highly sensitive detection is performed, without being affected by the coexistent DNA and background.

The method of this invention used in the microreactor is more specifically, performed by the following steps. In other words, the method of this invention is performed using the microreactor and includes

(1) a step of feeding the cDNA synthesized by a reverse transcription reaction by the specimen or the DNA extracted from the specimen, or alternatively the specimen or the RNA extracted from the specimen and a biotin modified primer from the respective storage section to the flow path and performing a gene amplification reaction in a flow path;

(2) a step of mixing the reaction solution including the amplified gene and the denaturant in the micro tubes and performing processing for denaturing the amplified gene into a single strand;

(3) a step of feeding the processing solution that has been processed for denaturing the amplified DNA to a single strand to a flow path to which streptavidin has been adsorbed and then and fixing the amplified gene;

(4) a step of flowing probe DNA whose end has undergone fluorescent marking with FITC (fluorescein isothiocyanate) into the minute flow path into which the amplified gene is fixed and hybridizing the fixed gene with the probe DNA;

(5) a step of flowing gold colloid whose surface has been modified with a FITC antibody which binds specifically with FITC into the minute flow path and adsorbing gold colloid to the probe; and

(6) a step of optically measuring the concentration of the gold colloid in the minute flow path.

Biotinophilic protein includes avidin, streptoavidin and extra-avidin (R). These forms of avidin each have four avidin binding sites. Of these, streptoavidin is preferred in particular, because it has a higher level of specificity with biotin, and ensures rigid bondage. Streptoavidin is adsorbed by applying the solution obtained by dissolving it in a buffer solution, to the portion for immobilization inside the minute flow path. The present inventors have clarified the suitable conditions for ensuring that the protein derived from streptomyces avidinii is adsorbed to the portion for immobilization inside the minute flow path. The details are disclosed in the embodiment to be described later. Surprisingly, no special chemical processing is required when the streptoavidin is immobilized inside the minute flow path formed on the plastic substrate. Namely, only the following steps are sufficient: The biotinophilic protein is dissolved in the SSC buffer solution or physiological saline solution to prepare a solution having a concentration of 10 through 35 μg/mL, preferably, 20 through 30 μg/mL. This is applied onto the minute flow path downstream of the amplification reaction site. Then biotinophilic protein is adsorbed on the flow path arranged on the plastic substrate. When the streptoavidin has been immobilized in the aforementioned manner, the detection site for trapping the amplified gene can be provided very easily. To increase the amount of the streptoavidin to be adsorbed, the polystyrene adsorption site may be provided with fine concavo-convex patterns, for example, filaments to increase the surface area of the detection site. Alternatively, it is also possible to prepare a separate minute plastic strip on which streptoavidin is adsorbed or immobilized, or to use a porous substance.

The fluorescent labeled oligodeoxynucleotide is preferably used as the probe DNA for gene inspection. The sequence complementary with part of the base sequence of the gene to be detected is selected as a DNA base sequence. A commonly known fluorescent pigment can be used as a fluorescent pigment for labeling the probe. For example, it contains fluorescent substrates such as common FITC, RITC (rhodamine isothiocyanate), NBD, Cy3 and Cy5. Particularly the FITC is preferred because anti-FITC antibody, for example, gold colloid anti-FITC antibody IgG can be obtained. Digoxigenin (DIG) of steroid hapten, instead of the fluorescent pigment, may be labeled with the probe DNA. In this case, an anti-DIG-alkali phosphatase labeled antibody is used as an alternative to the anti-FITC antibody.

The above-mentioned method includes the commonly known technologies of immobilization of biotin-introduced DNA and biotin-streptoavidin combination, designing of fluorophore labeled FITC, anti-FITC antibody, primer and probe, and production of the primer and probe. The hybridization of nucleic acid also belongs to the conventional art. The scale and efficiency heavily depend on various conditions. The above-mentioned description contains the method wherein the amplified gene is trapped by the streptoavidin through the biotin labeled to the primer and is hybridized with it. In some cases, the order can be reversed in such a way that the amplified gene is first hybridized with the probe DNA, and then the product resulting from hybridization is trapped by the streptoavidin through the biotin labeled to the prime. If the specific base sequence of the amplified gene trapped by the streptoavidin and the base sequence of the probe DNA are complementary to each other, satisfactory hybridization will result. In any case, converting the probe DNA into a coloring substance will ensure optical detection to be achieved by the detection apparatus of the DNA inspection device of the present invention.

The fluorescence of a fluorescent pigment FITC can also be measured. In this case, however, photofading and background noise of the fluorescent pigment must also be taken into account. It is preferred to use the method that permits highly sensitive measurement by final visible light. In the present invention, a gold colloid optical detection method based on the gold colloid anti-FITC mouse IgG is used. The visible light absorption spectroscopy allows use of more general purpose equipment and ensures less disturbing factors and easier data processing. Preferably, a step of feeding the washing solution in the flow path adsorbing the streptoavidin, is arranged between the aforementioned steps, wherever required. A preferred washing solution includes various types of buffer solutions, salts solution and organic solvent. In the aforementioned steps, the solution for modification is a reagent for forming gene DNA into one chain, and includes sodium hydroxide and potassium hydroxide, for example.

Alternatively, the aforementioned probe can be labeled with HRP (horseradish peroxidase, instead of the aforementioned fluorescent pigment. It is also possible to use the reaction of color development catalyzed by this enzyme. The commonly known color developing substance for this purpose includes 3,3′,5,5′-tetramethylbenzine (TMB), 3,3′-diaminobenzidine (DAB), p-phenylenediamine, 5-aminosalicylic acid (5AS), 3-amino-9-ethylcarbazole (AEC), 4-chloro-1-naphthol (4CIN), 4-amino anti-pyrine and o-dianisidine. Reaction is caused to take place between the coloring reagent containing any one of these substances and peroxidase of the probe so color development occurs to the substance.

Enzyme/color development system such as alkali phosphatase and galactosidase can also be used in addition to peroxidase.

Excellent features of visible absorption spectroscopy as described above are provided, as compared to fluorometry. This is because general purpose equipment can be used, disturbing factors is less numerous and data processing is easier. In the DNA inspection device of the present invention, the optical inspection apparatus together with the liquid feed section containing the above-mentioned micro-pump and the temperature control apparatus for controlling the temperature of each reaction in the flow path of the microreactor are preferably incorporated to form an integral structure.

Preferably, a step of feeding the washing solution in the flow path adsorbing the streptoavidin, is arranged between the aforementioned steps, wherever required. A preferred washing solution includes various types of buffer solutions, salts solution and organic solvent. In the aforementioned steps, the solution for modification is a reagent for forming gene DNA into one chain, and includes sodium hydroxide and potassium hydroxide, for example.

The present invention also includes the following method of creating the detection site of the gene inspection microreactor wherein, in order to ensure that the biotinophilic protein for trapping the gene amplified inside the minute flow path is immobilized, as a gene detection site, downstream of the gene amplification reaction site of the flow path, the biotinophilic protein is dissolved in a physiological saline solution or an SSC buffer to prepare a solution having a concentration of 10 through 35 μg/mL, and the solution is applied to the minute flow path formed with polystyrene, whereby biotinophilic protein is adsorbed on the minute flow path.

The method of detecting the amplified gene of the present invention includes the steps of:

modifying the amplified gene into one chain by mixing between the reaction solution containing the gene amplified at the gene amplification reaction site in the minute flow path of the microreactor, and the modification solution;

feeding the processing solution obtained by modification of the amplified gene into one chain, to the inspection site of the minute flow path wherein the biotinophilic protein is adsorbed onto the polystyrene so that the amplified gene is trapped by the detection site;

feeding the peroxidase-modified probe DNA to the detection site so as to hybridize the amplified gene with the probe DNA;

feeding the coloring reagent solution containing the coloring substance to the detection site trapping the amplified gene so that the coloring substance will be colored by the reaction catalyzed by peroxidase; and

optically measuring the coloring that takes place at the detection site.

In the aforementioned amplification and detection, the preset conditions on the order, capacity and timing in feeding the liquid as well as the micro-pump and temperature control are incorporated in the software of the biological substance inspection device in the form of a program. If the DNA inspection device proper integrally incorporating the software, micro-pump, detection apparatus and temperature control apparatus are linked with the microreactor removably mounted on this apparatus proper, then the flow path of the microreactor is activated. Preferably, analysis is automatically started. Reaction of the gene amplification resulting from feeding and mixing the sample and reagent, reaction of gene detection, and optical measurement are performed automatically in a series of continuous operation steps. Then the measurement data containing required conditions record items is stored into the file.

Inspection by Microreactor

Mainly two aspects in gene inspection are provided by the gene amplification method and hybridization method adopted as the detection method in the biological substance inspection device and microreactor of the present invention. A primer having a specific sequence in a certain gene is used as a primer used in the gene amplification reaction, whereby the presence or absence of amplification or amplification efficiency is measured. This makes it possible to determine if the DNA derived from the gene in the sample is the same as the special gene or is different from it. This method is effective especially in quick identification or determination of a virus or bacteria causing an infectious disease. A slight mutation between allelic genes on the homologous chromosome can be detected by the gene specific PCR that utilizes the aller-specific oligonucleotide as a PCR oligomer. This microreactor is also compatible with simultaneous measurement of a plurality of items. When a plurality of primers with the base arrangement changed as appropriate are prepared as the primers used in the gene inspection, the present microreactor can be used for identification and distinction of mutants in the bacteria and viruses of the same type.

The nucleotide sequence of the probe DNA hybridized with the amplified gene DNA is arranged to be complementary to the target gene, thereby improving the detection accuracy. Alternatively, it is also possible used to detect the gene variation wherein mismatching with synthetic probe in hybridization is used as an index.

The gene inspection by the present invention provides the data for diagnosing the degree of expression of high blood pressure gene. To put it more specifically, the gene inspection by the present invention makes it possible to analyze the type of the mRNA as a proof of the expression of such a gene and the level of expression.

Alternatively, gene inspection based on the microreactor of the present invention L determines of a genetic factor exhibiting the susceptibility to a specific disease, and detects genetic variations involving the adverse effect of medicine and variations in the area of regulating gene promoter in addition to coding area. In this case, the primer having a nucleic acid sequence containing a varied portion is used. The aforementioned genetic variation refers to the variation in the nucleotide base of the gene. Analysis of the gene polymorphism using the inspection apparatus of the present invention helps identify the gene susceptible to disease.

The configuration of the apparatus and the principle of analysis clearly indicate that various gene inspection methods based on the inspection apparatus of the present invention produce higher precision results in a shorter time with smaller effort using much smaller volume of the sample and simpler apparatus, than the conventional methods of nucleic acid sequence analysis, restriction enzyme analysis and nucleic acid hybridization analysis.

The biological substance inspection microreactor and biological substance inspection apparatus of the present invention can be used in the field of gene expression analysis, gene function analysis, single nucleotide polymorphic analysis (SNP), clinical examination/diagnosis, medicine screening, inspection for the safety and toxicity of medicine, agricultural chemical or various other chemicals, environmental analysis, food product inspection, inspection in the field of forensic medicine, chemistry, brewing, fishery, stockbreeding, production of farm products, agriculture, forestry, etc.

Referring to the drawing shown as an example of the preferred embodiment of the present invention, the following further describes the example of the gene inspection, without the present invention being restricted thereto.

The micro-reactor composed on one chip made of resin shown in FIG. 5 automatically performs gene amplification reaction and detection in the chip according to the ICAN method when by injected with the gene sample extracted from the blood or phlegm, whereby simultaneous diagnosis of a plurality of genes is performed. For example, about 2 through 3 μL of blood sample is dropped onto the chip having a length and width of several centimeters. This operation alone allows amplification reaction and detection to be performed when the chip is mounted on the apparatus proper 2 shown in FIG. 5.

The sample injected into the sample storage section 20 and the reagent used for the gene amplification reaction sealed in advance into the reagent storage sections 18a through 18c of FIG. 11 (including the biotin-modified hybrid primer that specifically hybridizes with the gene as an object of detection, the DNA polymerase of chain labilization, and the endonuclease) are fed to the flow path communicating with each storage section by the micro-pump (not illustrated) incorporated in the apparatus proper of FIG. 5. Then the sample and reagent are mixed in the flow path through the Y-shaped flow path, whereby amplification reaction is conducted. The minute flow path is formed to have a width of 100 μm and a depth of 100 μm, for example. The DNA amplified in this manner is detected by optically measuring the gold colloid at the concentration used for bonding. To put it more specifically, it is detected by the optical detection apparatus (not illustrated) incorporated into the apparatus proper 2 of FIG. 5. For example, light for measurement is applied to the detection site on the analysis flow path for each of the inspection item from the LED or others. The transmitted light or reflected light is detected by an optical detecting device such as an photodiode, CCD camera or photomultiplier tube, whereby the amplified DNA (gene) labeled through the DNA hybridized by this procedure is detected.

In the apparatus 2, a temperature control device to control a reaction temperature is incorporated so that gene inspection can be conducted simply by merely attaching a chip, in which a reagent has been incorporated, onto a small unit in which a liquid feeding pump, a optical detecting device and a temperature control device are made in one body.

In the present embodiment, the microreactor has the following structure to ensure that high-precision, high-speed and high-reliability gene inspection is conducted by one chip.

In the first place, all forms of control are integrated into one chip. The internal control, positive control and negative control are sealed into the microreactor in advance. The reagent is divided by the operation of the microreactor. Concurrently with the sample amplification reaction and detection operation, predetermined steps are taken for amplification reaction and detection of these forms of control. This arrangement allows high-speed and high-reliability gene inspection to be performed.

Secondly, the microreactor is provided with:

a liquid feed control section capable of controlling the passage of liquid by the micro-pump pressure, wherein the flow of liquid to each predetermined position of the flow path is blocked until the liquid feed pressure in the forward direction reaches a predetermined level, and the liquid feed pressure above the preset level is then added to allow passage of the liquid; and

a backflow preventing section for preventing the liquid in the flow path from back-flowing.

The flow of liquid in the flow path is controlled by the micro-pump, liquid feed control section and backflow preventing section. To be more specific, the reagent and sample are divided during the feed and a fixed amount of the reagent can be fed with high precision. Further, a plurality of reagents fed from the branched flow path can be mixed at a high speed.

The amplification reaction and detection operation using the microreactor of the present invention will be described with reference to the major components of the microreactor.

Reagent Storage Section

The microreactor 1 is provided with a plurality of reagent storage sections 18, which stores the reagent used for gene amplification reaction, the solution used for modification of the amplified gene and the probe DNA to be hybridized with the amplified gene.

The reagent storage section 18 is preferably loaded with reagent in advance so that the quick inspection can be conducted independently of the place or time. The surface of the reagent storage section is sealed to prevent the reagents incorporated in the chip from being subjected to evaporation, loss by leakage, entry of bubbles, contamination and deterioration. Further, when the microreactor is kept in store, it is filled with a sealant to ensure that the reagent will not leak from the reagent storage section into the minute flow path and reaction of the reagent will not occur. When the reagent is stored in the microreactor in advance, the microreactor is preferably kept in cold storage for the safety of reagent. This sealant is solidified or gelated before use under the cold-storage condition where the microreactor is stored. When its temperature is raised to the room temperature immediately before use, the sealant melts and becomes fluid. The reagent is preferably sealed into the reagent storage section by placing sealant between the reagent and flow path 15 communicating with the reagent storage section 18. Air may be present between the sealant and reagent, but the amount of air present is preferred to be sufficiently small (with respect to the amount of reagent) in order to feed a fixed amount of liquid.

A plastic substance that does not easily dissolved in water can be used as the sealant. Use of oils and fats having a solubility of 1% or less is preferred. Similarly, a sealant may be applied between the storage sections for positive control and negative control, and the flow path communicating therewith.

Micro-Pump and Element

In the present embodiment, the sample storage section 20, reagent storage section 18, positive control storage section 21h and negative control storage section 21i are each provided with a micro-pump 11 for feeding the liquids in these surface tensions. The micro-pump 11 is connected to the upstream side of the reagent storage section 18, and the driving solution is fed to the reagent storage section by the micro-pump 11, whereby the reagent is pushed out into the flow path and is fed. The micro-pump unit is incorporated into an apparatus proper (biological substance inspection device) separate from the microreactor. When the microreactor is mounted on the apparatus proper, it is connected from the pump connecting section 12 to the microreactor. As a micro-pump element, in addition to the pump connection section 12, it may be possible to provide also a pump feeding liquid operating section in the minute flow passage. In this case, the micro-pump element includes the pump connection section 12 and the pump feeding liquid operating section.

Feed Control Section and Reverse Flow Prevention Section

A plurality of feed control sections are provided in the flow path of the microreactor of this embodiment as shown in FIG. 9(a). The feed control section interrupts the passage of fluid pressure in the normal direction until a prescribed pressure is reached, and passage of the fluid is permitted when a pressure not less than the prescribed pressure is applied.

The microreactor of this embodiment includes a plurality of reverse flow prevention sections for preventing reverse flow of the fluid in the flow paths. The reverse flow prevention section has a check valve in which the flow path opening is closed by a valve element due to reverse flow pressure, or an active valve in which a valve element is pressed onto the flow path opening portion by a valve element deforming means to close the opening.

Reagent Amount Determining Section

Quantitative feed of reagent can be performed using the aforementioned liquid feed control section and backflow preventing section. In the reagent determining section, a predetermined amount of reagent is applied in the flow path (reagent-filled flow path 15b) between the backflow preventing section 16 and liquid feed control section 13d. Further, a branched flow path is provided, which branches off from the reagent-filled flow path 15b and communicates with the micro-pump 11 for feed the drive liquid. The variation in quantitative determination will be reduced by installing a large-capacity reservoir 17a in the reagent-filled flow path 15b.

In the step of reagent mixing, two types of reagent are mixed in a Y-shaped flow path. In this case, the mixing ration in the leading portion of the liquid flow is not stabilized even if simultaneous feeding of reagents is performed. To solve this problem, the liquid mixture is preferably fed to the next step after the mixing ratio has been stabilized, by discarding the leading portion of the liquid flow.

Gene Amplification Reaction Site

Such reagents as a biotin modified hybrid primer that hybridizes specifically with the gene as a target for detection, a DNA polymerase of chain labilization, an endonuclease are stored in the reagent storage sections 18a, 18b and 18c in FIG. 11. On the side upstream of each reagent storage section, a piezo-pump 11, incorporated in the apparatus proper, separate from the microreactor is connected by the pump connecting section 12. Reagents are fed by these pumps to the flow path 15a on the downstream side from each reagent storage section.

The flow path 15a, the flow path branched off from the flow path 15a, leading to the next step, and the liquid feed control sections 13a and 13b are configured in such a way as to discard the leading portion of the reagent mixture fed from each reagent storage section, and to feed the reagent mixture to the next step after stable mixing has been reached. Each reagent storage section stores a total of more than 7.5 μL of reagent. A total of 7.5 μL of reagent mixture subsequent to the process of discarding the leading portion is fed to the three branched flow paths 15b, 15c and 15d, the amount of reagent fed to each of the flow paths being 2.5 μL. The flow path 15b communicates with a reaction/detection system 22 (FIGS. 5 and 11) (reaction with sample); the flow path 15c with a reaction/detection system 22 (reaction with positive control); and the flow path 15d with the reaction/detection system 22 (reaction with negative control).

The reservoir 17a of FIG. 5 is filled in with the reagent mixture fed to the flow path 15b. A reagent-filled flow path is formed between the backflow preventing section 16 upstream of the reservoir 17a and the liquid feed control section 13d downstream thereof. It forms the aforementioned reagent determining section, together with the liquid feed control section 13e installed on the branched flow path communicating with the piezo-pump 11 for feeding drive liquid.

The sample extracted from the blood and phlegm is injected from the sample storage section 20 in FIG. 5. A fixed amount of sample (2.5 μL) is fed into the reservoir 17b using the same mechanism as that of the aforementioned reagent determining section, and is then fed to the succeeding flow path. The sample filling in each of the reservoirs 17 and the reagent mixture are fed to the flow path 15e (volume: 5 μL) through the Y-shaped flow path. Mixing and ICAN reaction are carried out in the flow path 15e. Here the sample and reagent are fed by the pumps 11 and 11b, which are alternately driven to introduce the round slices of sample and reagent mixture alternately into the flow path 15e, thereby ensuring quick diffusion and mixing between the simple and reagent.

In the amplification reaction, 5 μL of reaction solution and 1 μL of reaction stop solution stored in the stop solution storage section 21a are fed into the flow path 15f having a volume of 6 μL, and are mixed together, whereby amplification reaction is stopped. Then 1 μL of the modification solution stored in the modification solution storage section 21b and 0.5 μL of the mixture of reaction solution and stop solution are fed to the flow path 15g having a volume of 1.5 μL, and are mixed. The amplified gene is modified into one chain. Then 2.5 μL of the hybridization buffer stored in the hybridization buffer storage section 21c and 1.5 μL of processing solution having been modified are fed to the flow path 15h having a volume of 4 μL, where they are mixed there.

Detection Site

The processing solution is fed to the detection sites 22a and 22b with streptoavidin adsorbed inside the flow path, the amount fed each time being 2 μL. The aforementioned amplified gene is immobilized in this flow path. The washing solution stored in each of the storage sections 21d, 21f and 21e, the probe DNA solution with the terminus fluorescent-labeled with the FITC, and gold colloid labeled with the anti-FITS antibody are fed by the single pump 11 into the flow path 22a where this amplified gene is immobilized, in the order illustrated in FIG. 11. At the same time, the washing solution stored in each of the storage sections 21d, 21g and 21e, the probe DNA solution for internal control, and gold colloid labeled with the anti-FITS antibody are fed by the single pump 11 into the flow path 22b where the amplified gene is immobilized, in the order illustrated in the same figure. Then the probe DNA is immobilized with the amplified gene of one chain having been immobilized. A required washing solution is loaded into the washing solution storage section 21d, as appropriate.

When the gold colloid solution is fed, gold colloid is bonded with the immobilized amplified gene through the FITC of the probe DNA, and is immobilized in position. The presence or absence of amplification or amplification efficiency is identified by optical detection of the immobilized gold colloid.

The flow paths 15c and 15d communicates with the positive control reaction/detection system and negative control reaction/detection system. Similarly to the case of the aforementioned sample reaction/detection system, the reagent mixture is fed to these paths, and amplification reaction is conducted with the sample in the flow path. After that, the reagent mixture is hybridized with the probe DNA stored in the probe DNA storage section. Then the amplification reaction is detected based on the reaction product.

### Embodiment

The following describes the present invention in greater details with reference to the embodiment. It should be noted, however, that the present invention is not restricted thereto.

Reagents Used

Streptoavidin: by Nakalaytesque Inc.
Biotin-introduced gold colloid: Albumin-biotin gold labeled, 20 nm (by Sigma Inc., Product No. A4417)

This was subjected to 50-fold dilution using the following 5×SSC:
Buffer solution (Infiltrated with a 0.2 μm filter for sterilization after preparation)

5×SSC: 750 mM sodium chloride and 75 mM trisodium citrate

Physiological saline solution: 0.9% sodium chloride

50 mM tris-HCl; Tris refers to 2-amino-2-hydroxymethyl-1,3-propandiol.

Pure water

Detection

A light emitting diode having a maximum wavelength of 520 through 530 nm was placed opposite to a photodiode, and the portion of the sample to be measured was placed between them to measure the photodiode output. To be more specific, the adsorption intensity can be expressed by the following equation:
log (I₀/(I_{g}−I_{b}))

where “I₀” denotes the numerical value when there was nothing between the light emitting diode and photodiode, “I_{b}” the numerical value on an original basis when not adsorbed, and “I_{g}” the numerical value when the gold colloid is reacted.

Procedure

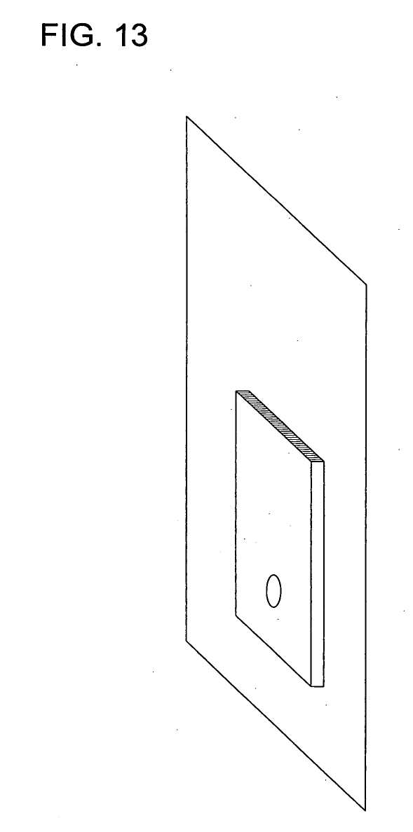

As shown in FIG. 13, a silicone-rubber with holes each having a diameter of 4 mm was bonded on a transparent plastic sheet. These holes each were filled with 12 μL of streptoavidin solutions having various concentrations (9 concentrations ranging from 10 through 50 μg/mL), prepared using various types of buffer solutions (Tris buffer, SSC buffer, hybrid buffer, and physiological saline solution). Silicone rubber covers were placed over the holes of silicone rubber to block them. They were left to stand for an hour at the room temperature. The streptoavidin solution was removed and the holes are washed three times by various types of buffer solution. Then 2 μL of biotin-labeled gold colloid was put into the silicone holes. The biotin-labeled gold colloid was removed and the holes are washed three times by various types of buffer solution. The silicone rubber was removed and the polystyrene sheet was dried.

After that, the optical concentration on the hole portion of the transparent plastic sheet and other portions was measured and the adsorption intensity of the gold colloid was calculated according to the aforementioned expression. Table 1 shows the result obtained by this procedure. It has been revealed that the optimum streptoavidin concentration is 25 μg/mL. The buffer solutions to be used include physiological saline solution, SSC Tris and pure water in that order of preference.

TABLE 1
Streptoavidin adsorption intensity

| Streptoavidin | | | | |
|---|---|---|---|---|
| concentration | | | | Physiological |
| (μg/mL) | Tris | Pure water | 5 × SSC | saline solution |
| 10 | 0.0006 | 0.0006 | 0.0033 | 0.0034 |
| 15 | 0.0009 | 0.0009 | 0.0034 | 0.0176 |
| 20 | 0.0005 | 0.0005 | 0.0139 | 0.0275 |
| 25 | 0.0009 | 0.0009 | 0.0261 | 0.0517 |
| 30 | 0.0013 | 0.0003 | 0.0191 | 0.0157 |
| 35 | 0.0084 | 0.0024 | 0.0183 | 0.0097 |
| 40 | 0.0021 | 0.0021 | 0.0145 | 0.0102 |
| 45 | 0.0015 | 0.0015 | 0.0125 | 0.0048 |
| 50 | 0.0008 | 0.0008 | 0.0127 | 0.0096 |

As mentioned above, although the genetic screening was explained with the main example, referring to the drawing shown as an example of the desirable embodiment of the present invention, the present invention is not limited to these embodiments and example.

The micro reactor of the present invention for genetic screening is aplicable in fields, such as a gene finding analysis, a gene performance analysis, 1 gene polymorphic analyze (SNP), a clinical test and a diagnosis, medicine screening, medicine, a safety and toxic examination of a pesticide or various chemical matters, an environmental assay test, a food evaluation, a forensic medicine, a chemistry, a brewing, a fishing, stock raising, an agricultural production, and agricultural and forestry industries.

What is claimed is:

1. A micro reactor for inspecting a biological material, comprising:
(1) a first substrate on which a minute flow path is formed;
(2) a second substrate laminated on the first substrate so as to cover the minute flow path;
(3) a detection section provided on the minute flow path formed between the first and second substrates so as to inspect a liquid mixture of a sample and a reagent;
(4) an opening section formed at a region on the second substrate corresponding to the detection section so as to irradiate inspection light to the liquid mixture arriving the detection section; and
(5) a transparent member to cover the opening section.

2. The micro reactor of claim 1, wherein the transparent member is a film, at least the outer periphery of the opening of the second substrate is colored, and the film is heat-sealed on the first substrate by applying heat to this colored area from the film side with a laser.

3. The micro reactor of claim 1, wherein the detection section comprises a land section provided to reduce the depth of the minute flow path.

4. The micro reactor of claim 1, wherein a biotinophilic protein to trap gene amplified in the minute flow path is adsorbed on the detection section.

5. The micro reactor of claim 4, wherein the biotinophilic protein bonds with biotin labeled with 5′-terminus of a primer used in a gene amplification reaction.

6. The micro reactor of claim 4, wherein the biotinophilic is streptoavidin.

7. The micro reactor of claim 4, wherein the streptoavidin is adsorbed by applying a solution obtained by dissolving the streptoavidin in a buffer solution to the detecting section.

8. The micro reactor of claim 7, wherein a concentration of the streptoavidin in the solution is 10 through 35 μg/mL.

9. The micro reactor of claim 7, wherein the buffer solution is a physiological saline solution or an SSC buffer.

10. The micro reactor of claim 4, wherein the amplification reaction is performed according to an ICAN (registered trademark) method.

11. The micro reactor of claim 10, wherein the probe to be hybridized with the amplified gene is labeled with fluorescent pigment.

12. The micro reactor of claim 11, wherein the fluorescent pigment preferably is a FITC.

13. The micro reactor of claim 12, wherein the probe is colored with gold colloid modified by the anti-FITC antibody.

14. The micro reactor of claim 1, wherein biotinophilic protein is absorbed by polystyrene at the detection section to trap a sample gene amplified by a biotin-labeled primer.

15. The micro reactor of claim 14, wherein a probe to be hybridized with the amplified gene is labeled with peroxidase.

16. The micro reactor of claim 15, wherein the probe is made to react with a coloring reagent.

17. The micro reactor of claim 16, wherein the coloring reagent includes as a coloring substance 3,3′,5,5′-tetramethyl benzidine, 3,3′-diamino benzidine, p-phenylene diamine, 5-amino salicylic acid, 3-amino-9-ethylcarbazole, 4-chloro-1-naphthol, 4-amino antipyrine or o-dianisidine.

18. The micro reactor of claim 1, wherein the second substrate to cover the minute flow path and the detection section provide on the first substrate is transparent.

19. The micro reactor of claim 18, wherein a transparent material of the second substrate is polystyrene, a PE, polypropylene, a polyethylene terephthalate, polyethylenenaphthalate, polymethylmethacrylate, polyethylene vinyl alcohol, acryl resin, polyvinyl resin, epoxy resin, polyvinyl chloride, a unsaturated polyester resin, polyamide resin, polyimide resin, polysulfone resin, annular cycloolefin resin, cellulose acetate, cellulose nitrate, fluorocarbon resin, polycarbonate, or poly dimethyl siloxane.

20. The micro reactor of claim 18, wherein a material of the first substrate is polystyrene.

21. The micro reactor of claim 18, wherein an inner surface of the minute flow path is coated with protein.

22. The micro reactor of claim 1, wherein the minute flow path is a minute flow path branched into at least two or more paths, a fluid containing specimen is fed to downstream point in each branched micro flow path and/or a reagent encapsulated or its mixture fluid are fed to downstream points in each branched micro flow path by a micro pump and a feeding fluid dividing means, and a plurality of items and/or controls can be measured simultaneously at downstream points of each branched micro flow path.

23. The micro reactor of claim 22, wherein the micro pump is a piezo pump provided with a first flow path whose flow resistance varies with the change of pressure difference, a second flow path in which a ratio of a change of flow resistance to a change of pressure difference is smaller than that of the first flow path, a pressure chamber connected to both the first flow path and the second flow path, and with an actuator which changes the pressure inside the pressure chamber.

24. The micro reactor of claim 22, wherein the feeding fluid dividing means comprises branched micro flow paths, a feeding fluid control section able to control the passage of the fluid though the pump pressure of the micro pump that intercepts passage of the fluid until the fluid feeding pressure of positive direction reaches to a predetermined pressure and allows passage by applying fluid feeding pressure exceeding the predetermined pressure, and a backward flow preventing section which prevents backward flow in the flow path, and controls feeding of feeding fluid, an fixed amount of feeding fluid and mixing of each fluid in the divided flow path.

25. The micro reactor of claim 1, wherein a substrate is further adhered as a bottom substrate to cover a concave formed on the bottom of the first substrate so as to form a waste fluid reservoir section to reserve waste fluid resulting from condensing, washing and measuring of the specimen.

26. The micro reactor of claim 1, wherein a viscosity of fluid flowing through the minute flow path is controlled to be not more than 10 mPa·s.
